(19) United States
(12) Patent Application Publication
(10) Pub. No.: US 2023/0218198 A1
(43) Pub. Date: Jul. 13, 2023

(54) METHODS AND SYSTEMS FOR NEUROFEEDBACK TRAINING

(71) Applicant: Synchron Australia Pty Limited, Melbourne (AU)

(72) Inventors: James BENNETT, Elwood (AU); Peter Eli YOO, Brooklyn, NY (US); Nicholas Lachlan OPIE, Melbourne (AU)

(73) Assignee: Synchron Australia Pty Limited, Melbourne (AU)

(21) Appl. No.: 18/153,972

(22) Filed: Jan. 12, 2023

Related U.S. Application Data
(60) Provisional application No. 63/266,755, filed on Jan. 13, 2022.

(51) Int. Cl. A61B 5/11; A61B 5/00

(52) U.S. Cl. CPC A61B 5/11; A61B 5/681

(57) ABSTRACT

Systems and methods for neurofeedback training are disclosed. In some embodiments, a brain-computing interface system comprises a recording device configured to record a brain activity of a subject and a computing device communicatively coupled to the recording device. The computing device can comprise one or more processors programmed to generate at least one of an auditory feedback, tactile feedback, and a neurofeedback graphical user interface (GUI) comprising a plurality of graphics representing the brain activity of the subject. The neurofeedback GUI, the auditory feedback, and the tactile feedback can aid the subject in producing brain activity that align with a desired intention of the subject.

### CROSS-REFERENCE TO RELATED APPLICATION

This application claims the benefit of U.S. Provisional Application No. 63/266,755 filed on Jan. 13, 2022, the content of which is hereby incorporated by reference in its entirety.

### TECHNICAL FIELD

This disclosure relates generally to brain-computer interfaces and, more specifically, to methods and systems for neurofeedback training for brain-computer interfaces (BCIs).

### BACKGROUND

Neurofeedback is a form of biofeedback that refers to providing a representation of a subject's own neural activity to the subject to aid the subject in self-regulating the relevant neural components (e.g., the cortical regions, nerves, etc.) that control a particular behavior. For example, subjects can volitionally regulate the amount of activity in their left and right sensorimotor cortices by attempting, or formulating thoughts concerning, right- and left-hand movements, respectively. These neural components, specifically, the frequency changes in certain cortical regions, are commonly used to control BCIs. Neurofeedback can, therefore, be utilized to improve BCI performance and, in the case of online BCIs, is likely a necessity or unavoidable.

Operant conditioning via immediate neurofeedback has been shown to enhance the signal-to-noise ratio after extended periods of training with BCIs (see Wolpaw, Jonathan R., and Dennis J. McFarland. “Control of a two-dimensional movement signal by a noninvasive brain-computer interface in humans.” Proceedings of the National Academy of Sciences 101.51 (2004): 17849-17854; McFarland, Dennis J., et al. “Brain-computer interface (BCI) operation: signal and noise during early training sessions.” Clinical Neurophysiology 116.1 (2005): 56-62; and Pichiorri, Floriana, et al. “Sensorimotor rhythm-based brain—computer interface training: the impact on motor cortical responsiveness.” Journal of Neural Engineering 8.2 (2011): 025020). However, neural signals acquired by BCIs are usually multivariate and difficult to succinctly visualize. There are many aspects of the data that can be shown to the subject but, in most cases, only a subset of these aspects will be useful for enhancing the subject's control over their own neural activity (e.g., enhancing sensorimotor rhythm modulations). For example, most BCIs record information from multiple electrodes, typically 16 to 64. For each electrode, there are likely many frequency bands that contain useful information regarding the subject's intention to perform a movement. This may include standard frequency bands like the mu, beta, and gamma bands as well as user-specific bands. The combination of electrodes and frequency bands gives rise to many (>100) potential markers of neural activity, termed “features” that may be useful to show the user. Frequency values can also be calculated across varying time windows which give rise to another dimension of possible features. However, the representation of neural activity shown to the user needs to be easy to understand. Even presenting the user with a subset of the most informative features can potentially overwhelm the subject.

Therefore, methods and systems are needed which can improve the accuracy and responsiveness of BCIs. Such methods and systems should be easy for a subject to grasp and not overwhelm the subject. Moreover, such methods and systems should be engaging and assist the subject in more effectively regulating their neural activity to better control the subject's BCI.

### SUMMARY

Systems and methods for neurofeedback training for brain-computer interfaces (BCIs) are disclosed. In some embodiments, a BCI system can comprise a recording device configured to record a brain activity of a subject; a computing device communicatively coupled to the recording device, wherein the computing device includes one or more processors programmed to: construct a neurofeedback graphical user interface (GUI) including a plurality of graphic portions including at least a first graphic portion representing a first intention of the subject calibrated to certain previously recorded brain activity of the subject and a second graphic portion representing a second intention of the subject calibrated to certain other previously recorded brain activity of the subject, and construct a graphic element appearing in at least one of the first graphic portion and the second graphic portion, wherein the graphic element represents a current brain activity of the subject recorded by the recording device, and wherein the graphic element is moveable between the first graphic portion and the second graphic portion; and a display, communicatively coupled to the computing device, configured to display the neurofeedback GUI and the graphic element to the subject to aid the subject in producing brain activity that align with a desired intention of the subject.

In certain embodiments, the desired intention is one of the first intention or the second intention. In some embodiments, the first intention and the second intention are not related to viewing the plurality of graphic portions or the subject focusing their attention on the plurality of graphic portions.

In certain embodiments, the brain activity of the subject recorded by the recording device is reduced to univariate data, wherein the univariate data is presented in a one-dimensional space through the neurofeedback GUI, wherein the first graphic portion includes a first segment of the one-dimensional space, and wherein the second graphic portion includes a second segment of the one-dimensional space.

In certain embodiments, the one-dimensional space is a number line and the graphic element is a dot moveable along the number line.

In certain embodiments, the brain activity of the subject recorded by the recording device is collected as multivariate data, wherein the multivariate data is presented in a two-dimensional space through the neurofeedback GUI, wherein the first graphic portion includes a first area of the two-dimensional space, and wherein the second graphic portion includes a second area of the two-dimensional space.

In certain embodiments, the two-dimensional space is a graph having two axes.

In certain embodiments, the multivariate data is reduced into the two-dimensional space using a dimensionality reduction function. In certain embodiments, the multivariate data is reduced into the two-dimensional space using principal component analysis (PCA).

In certain embodiments, the first area and the second area of the two-dimensional space are positioned on the graph by projecting coordinates within the two-dimensional space back to a multi-dimensional space corresponding to the multivariate data.

In certain embodiments, the multivariate data is reduced into the two-dimensional space using a hyperplane of a linear classifier.

In certain embodiments, the multivariate data is reduced into the two-dimensional space using a non-linear dimensionality reduction function.

In certain embodiments, the graphic element is a dot moveable within the graph.

In certain embodiments, the first graphic portion is presented in a first color and the second graphic portion is presented in a second color.

In certain embodiments, the brain activity recorded is a power of a neural oscillation or brainwave of the subject or a change in blood flow within the brain of the subject.

In certain embodiments, the recording device is at least one of an implantable recording device, an electroencephalography (EEG) device, an electrocorticography (ECoG) device, a functional magnetic resonance imaging (fMRI) machine, and a functional near-infrared spectroscopy (fNIRS) device.

In certain embodiments, either the first intention or the second intention is an intention of the subject to move a body part of the subject. More specifically, the first intention and the second intention are not related to viewing the plurality of graphic portions or the subject focusing their attention on the plurality of graphic portions.

In certain embodiments, either the first intention or the second intention is achieving or maintaining a neural rest state.

In certain embodiments, a size of the graphic element displayed is configured to change in accordance with a certainty value associated with a positioning of the graphic element within at least one of the first graphic portion and the second graphic portion based on the current brain activity of the subject recorded by the recording device.

In certain embodiments, a color intensity of the graphic element displayed is configured to change in accordance with a certainty value associated with a positioning of the graphic element within at least one of the first graphic portion and the second graphic portion based on the current brain activity of the subject recorded by the recording device.

In certain embodiments, the graphic element includes a plurality of smaller graphic icons and wherein a density or shape of the smaller graphic icons are configured to change in accordance with a certainty value associated with a positioning of the graphic element within at least one of the first graphic portion and the second graphic portion based on the current brain activity of the subject recorded by the recording device.

In some embodiments, the BCI system can comprise a recording device configured to record a brain activity of a subject; a computing device communicatively coupled to the recording device, wherein the computing device includes one or more processors programmed to: associate a first graphic element with a first intention of the subject calibrated to certain previously recorded brain activity of the subject and associate a second graphic element with a second intention of the subject calibrated to certain previously recorded brain activity of the subject, wherein the first graphic element is visually distinct from the second graphic element; a display, communicatively coupled to the computing device, configured to display instances of either the first graphic element or the second graphic element in temporal succession based on a current brain activity of the subject recorded by the recording device in order to aid the subject in producing brain activity that align with a desired intention of the subject, wherein the desired intention is either the first intention or the second intention.

In certain embodiments, the one or more processors of the computing device are further programmed to render the instances of the first graphic element or the second graphic element as filling up a container graphic rendered on the display.

In certain embodiments, the one or more processors of the computing device are further programmed to render the instances of the first graphic element or the second graphic element as filling up the container graphic from bottom to top.

In certain embodiments, the display is configured to display an instruction to the subject to formulate or carry out the desired intention of the subject.

In some embodiments, the BCI system can comprise: a recording device configured to record a brain activity of a subject; a computing device communicatively coupled to the recording device, wherein the computing device includes one or more processors programmed to: associate a first sound with a first intention of the subject calibrated to certain previously recorded brain activity of the subject and associate a second sound with a second intention of the subject calibrated to certain previously recorded brain activity of the subject, wherein the first sound is auditorily distinct from the second sound; a user output device, communicatively coupled to the computing device, configured to generate a user output to instruct the subject to formulate or carry out a desired intention of the subject, wherein the desired intention is either the first intention or the second intention; an auditory component, communicatively coupled to the computing device, configured to generate either the first sound or the second sound based on a current brain activity of the subject recorded by the recording device in order to aid the subject in producing brain activity that align with the desired intention of the subject.

In certain embodiments, the user output device is at least one of: the auditory component and wherein the user output includes an auditory instruction generated by the auditory component; and a display and wherein the user output includes a text instruction or other type of visual instruction rendered via the display.

In certain embodiments, the first sound has a first pitch and the second sound has a second pitch, and wherein the first pitch is different than the second pitch.

In some embodiments, the BCI system can comprise: a recording device configured to record a brain activity of a subject; a computing device communicatively coupled to the recording device, wherein the computing device includes one or more processors programmed to: associate a first tactile feedback with a first intention of the subject calibrated to certain previously recorded brain activity of the subject and associate a second tactile feedback with a second intention of the subject calibrated to certain previously recorded brain activity of the subject, wherein the first tactile feedback is sensorially distinct from the second tactile feedback; a user output device, communicatively coupled to the computing device, configured to generate a user output to instruct the subject to formulate or carry out a desired intention of the subject, wherein the desired intention is either the first intention or the second intention; a tactile feedback component, communicatively coupled to the computing device, configured to generate either the first tactile feedback or the second tactile feedback based on a current brain activity of the subject recorded by the recording device in order to aid the subject in producing brain activity that align with the desired intention of the subject.

In certain embodiments, the user output device is at least one of: an auditory component and wherein the user output includes an auditory instruction generated by the auditory component; and a display and wherein the user output includes a text instruction or other type of visual instruction rendered via the display.

In certain embodiments, the first tactile feedback has a first vibratory frequency and the second tactile feedback has a second vibratory frequency, and wherein the first vibratory frequency is different than the second vibratory frequency.

In some embodiments, a method of conducting neurofeedback training is also disclosed. The method can comprise: recording a brain activity of a subject using a recording device; constructing a neurofeedback graphical user interface (GUI) using one or more processors of a computing device communicatively coupled to the recording device, wherein the neurofeedback GUI includes a plurality of graphic portions including at least a first graphic portion representing a first intention of the subject calibrated to certain previously recorded brain activity of the subject and a second graphic portion representing a second intention of the subject calibrated to certain other previously recorded brain activity of the subject; constructing a graphic element appearing in at least one of the first graphic portion and the second graphic portion, wherein the graphic element represents a current brain activity of the subject recorded by the recording device, and wherein the graphic element is moveable between the first graphic portion and the second graphic portion; and displaying the neurofeedback GUI and the graphic element to the subject via a display communicatively coupled to the computing device to aid the subject in producing brain activity that align with a desired intention of the subject.

In certain embodiments, the desired intention is one of the first intention or the second intention.

In certain embodiments, the brain activity of the subject recorded by the recording device is reduced to univariate data, wherein the univariate data is presented in a one-dimensional space through the neurofeedback GUI, wherein the first graphic portion includes a first segment of the one-dimensional space, and wherein the second graphic portion includes a second segment of the one-dimensional space.

In certain embodiments, the one-dimensional space is a number line and the graphic element is a dot moveable along the number line.

In certain embodiments, the brain activity of the subject recorded by the recording device is collected as multivariate data, wherein the multivariate data is presented in a two-dimensional space through the neurofeedback GUI, wherein the first graphic portion includes a first area of the two-dimensional space, and wherein the second graphic portion includes a second area of the two-dimensional space.

In certain embodiments, the two-dimensional space is a graph having two axes.

In certain embodiments, the multivariate data is reduced into the two-dimensional space using a dimensionality reduction function.

In certain embodiments, the multivariate data is reduced into the two-dimensional space using principal component analysis (PCA).

In certain embodiments, the first area and the second area of the two-dimensional space are positioned on the graph by projecting coordinates within the two-dimensional space back to a multi-dimensional space corresponding to the multivariate data.

In certain embodiments, the multivariate data is reduced into the two-dimensional space using a hyperplane of a linear classifier.

In certain embodiments, the multivariate data is reduced into the two-dimensional space using a non-linear dimensionality reduction function.

In certain embodiments, the graphic element is a dot moveable within the graph.

In certain embodiments, the first graphic portion is presented in a first color and the second graphic portion is presented in a second color.

In certain embodiments, the brain activity recorded is a power of a neural oscillation or brainwave of the subject or a change in blood flow within the brain of the subject.

In certain embodiments, the recording device is at least one of an implantable recording device, an electroencephalography (EEG) device, an electrocorticography (ECoG) device, a functional magnetic resonance imaging (fMRI) machine, and a functional near-infrared spectroscopy (fNIRS) device.

In certain embodiments, either the first intention or the second intention is an intention of the subject to move a body part of the subject. More specifically, the first intention and the second intention are not related to viewing the plurality of graphic portions or the subject focusing their attention on the plurality of graphic portions.

In certain embodiments, either the first intention or the second intention is achieving or maintaining a neural rest state.

In certain embodiments, a size of the graphic element displayed is configured to change in accordance with a certainty value associated with a positioning of the graphic element within at least one of the first graphic portion and the second graphic portion based on the current brain activity of the subject recorded by the recording device.

In certain embodiments, a color intensity of the graphic element displayed is configured to change in accordance with a certainty value associated with a positioning of the graphic element within at least one of the first graphic portion and the second graphic portion based on the current brain activity of the subject recorded by the recording device.

In certain embodiments, the graphic element includes a plurality of smaller graphic icons and wherein a density and shape of the smaller graphic icons are configured to change in accordance with a certainty value associated with a positioning of the graphic element within at least one of the first graphic portion and the second graphic portion based on the current brain activity of the subject recorded by the recording device.

Another method of conducting neurofeedback training is also disclosed. The method can comprise: recording a brain activity of a subject using a recording device; associating, using one or more processors of a computing device communicatively coupled to the recording device, a first graphic element with a first intention of the subject calibrated to certain previously recorded brain activity of the subject and associate a second graphic element with a second intention of the subject calibrated to certain previously recorded brain activity of the subject, wherein the first graphic element is visually distinct from the second graphic element; constructing a neurofeedback graphical user interface (GUI) using the one or more processors of the computing device; displaying, via a display communicatively coupled to the computing device, the neurofeedback GUI, wherein instances of either the first graphic element or the second graphic element are rendered in temporal succession as part of the neurofeedback GUI based on a current brain activity of the subject recorded by the recording device in order to aid the subject in producing brain activity that align with a desired intention of the subject, wherein the desired intention is either the first intention or the second intention.

In certain embodiments, the method further comprises rendering the instances of the first graphic element or the second graphic element as filling up a container graphic rendered as part of the neurofeedback GUI.

In certain embodiments, the method further comprises rendering the instances of the first graphic element or the second graphic element as filling up the container graphic from bottom to top.

In certain embodiments, the method further comprises displaying an instruction to the subject to formulate or carry out the desired intention of the subject via the display.

Yet another method of conducting neurofeedback training is disclosed. The method can comprise: recording a brain activity of a subject using a recording device; associating, using one or more processors of a computing device communicatively coupled to the recording device, a first sound with a first intention of the subject calibrated to certain previously recorded brain activity of the subject and associate a second sound with a second intention of the subject calibrated to certain previously recorded brain activity of the subject, wherein the first sound is auditorily distinct from the second sound; generating, using a user output device communicatively coupled to the computing device, a user output to instruct the subject to formulate or carry out a desired intention of the subject, wherein the desired intention is either the first intention or the second intention; generating, using an auditory component communicatively coupled to the computing device, either the first sound or the second sound based on a current brain activity of the subject recorded by the recording device in order to aid the subject in producing brain activity that align with the desired intention of the subject.

In certain embodiments, the user output device is at least one of: the auditory component and wherein the user output includes an auditory instruction generated by the auditory component; and a display and wherein the user output includes a text instruction or other type of visual instruction rendered via the display.

In certain embodiments, the first sound has a first pitch and the second sound has a second pitch, and wherein the first pitch is different than the second pitch.

An additional method of conducting neurofeedback training is also disclosed. The method can comprise: recording a brain activity of a subject using a recording device; associating, using one or more processors of a computing device communicatively coupled to the recording device, a first tactile feedback with a first intention of the subject calibrated to certain previously recorded brain activity of the subject and associate a second tactile feedback with a second intention of the subject calibrated to certain previously recorded brain activity of the subject, wherein the first tactile feedback is sensorially distinct from the second tactile feedback; generating, using a user output device communicatively coupled to the computing device, a user output to instruct the subject to formulate or carry out a desired intention of the subject, wherein the desired intention is either the first intention or the second intention; generating, using a tactile feedback component communicatively coupled to the computing device, either the first tactile feedback or the second tactile feedback based on a current brain activity of the subject recorded by the recording device in order to aid the subject in producing brain activity that align with the desired intention of the subject.

In certain embodiments, the user output device is at least one of: the auditory component and wherein the user output includes an auditory instruction generated by the auditory component; and a display and wherein the user output includes a text instruction or other type of visual instruction rendered via the display.

In certain embodiments, the first tactile feedback has a first vibratory frequency and the second tactile feedback has a second vibratory frequency, and wherein the first vibratory frequency is different than the second vibratory frequency.

### BRIEF DESCRIPTION OF THE DRAWINGS

The drawings shown and described are exemplary embodiments and non-limiting. Like reference numerals indicate identical or functionally equivalent features throughout.

FIG. 1A illustrates one embodiment of a brain-computer interface (BCI) system.

FIG. 1B illustrates one embodiment of a stent-electrode array implanted within a brain vessel of a subject. The stent-electrode array can be one example of a recording device of the BCI system.

FIG. 1C illustrates a communication conduit connecting the stent-electrode array with a telemetry unit of the BCI system.

FIG. 1D illustrates a close-up view of an embodiment of the telemetry unit of the BCI system.

FIG. 2A illustrates an embodiment of a coiled wire carrying a plurality of electrodes. The coiled wire can be another example of the recording device of the BCI system.

FIG. 2B illustrates an embodiment of an anchored wire carrying a plurality of electrodes. The anchored wire can be another example of the recording device of the BCI system.

FIG. 2C illustrates one embodiment of an electroencephalogram (EEG) device serving as the recording device of the BCI system.

FIG. 2D illustrates one embodiment of an electrocorticography (ECoG) device serving as the recording device of the BCI system.

FIG. 2E illustrates one embodiment of a functional magnetic resonance imaging (fMRI) device serving as the recording device of the BCI system.

FIG. 2F illustrates one embodiment of a functional near-infrared spectroscopy (fNIRS) device serving as the recording device of the BCI system.

FIG. 3 illustrates certain software layers or modules running on a computing device of the BCI system.

FIG. 4A illustrates one embodiment of a neurofeedback graphical user interface (GUI).

FIG. 4B illustrates another embodiment of the neurofeedback GUI.

FIG. 4C illustrates yet another embodiment of the neurofeedback GUI.

FIG. 4D illustrates an additional embodiment of the neurofeedback GUI.

FIG. 4E illustrates a further embodiment of the neurofeedback GUI.

FIG. 5A illustrates one embodiment of a graphic element displayed to the subject representing a current brain activity of the subject.

FIG. 5B illustrates another embodiment of a graphic element displayed to the subject representing a current brain activity of the subject.

FIG. 5C illustrates yet another embodiment of a graphic element displayed to the subject representing a current brain activity of the subject.

FIG. 6 illustrates an embodiment of a neurofeedback GUI comprising graphic elements displayed in temporal succession.

FIG. 7 illustrates examples of different types of sounds that can be generated by an auditory feedback component.

FIG. 8 illustrates examples of different types of tactile feedback components that can generate tactile feedback that can be felt by the subject.

### DETAILED DESCRIPTION

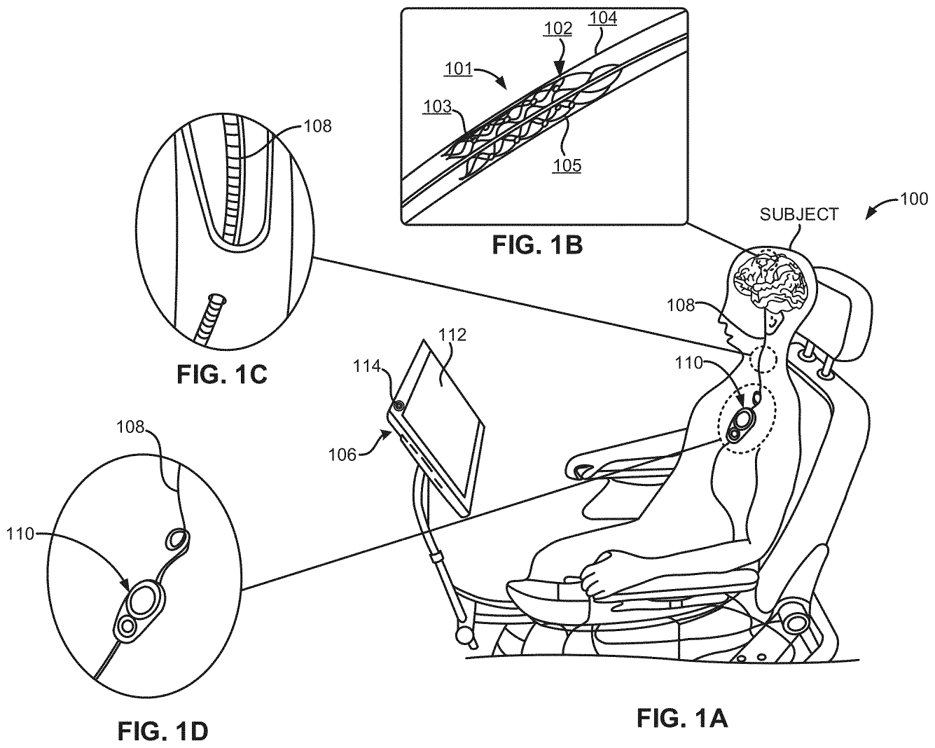

FIG. 1A illustrates one embodiment of a brain-computer interface (BCI) system 100 configured for neurofeedback training. As previously discussed, neurofeedback training can assist users of BCI systems to better control their BCI systems.

BCI systems are often used by subjects with mobility limitations to control peripheral devices such as personal electronic devices, internet of things (IoT) devices, or mobility vehicles or software applications running on such peripheral devices. An effective BCI system should allow the entire spectrum of subjects with mobility limitations to effectively control such peripheral devices or software applications, including those with severe mobility limitations such as locked-in subjects. Subjects control a BCI system by regulating their brain activity, which is monitored by one or more components of the BCI system.

The BCI system 100 disclosed herein provides neurofeedback training in a manner that does not overwhelm or confuse the subject. The system 100 is easy to comprehend and those that undergo the training procedure can improve their control over the BCI and, ultimately, improve their control of peripheral devices or software applications running on such peripheral devices communicatively coupled to the BCI.

The system 100 can comprise a recording device 101 (see FIG. 1B) and a computing device 106. The recording device 101 can be configured to record a brain activity of the subject. In some embodiments, the recording device 101 can be an invasive recording device 101 configured to be implanted within a brain vessel 104 of the subject.

For example, the recording device 101 can be a stent-electrode array 102 configured to be implanted within a brain vessel 104 of the subject (see, e.g., FIG. 1B). As a more specific example, the recording device 101 can be implanted within a cortical or cerebral vein or sinus of the subject.

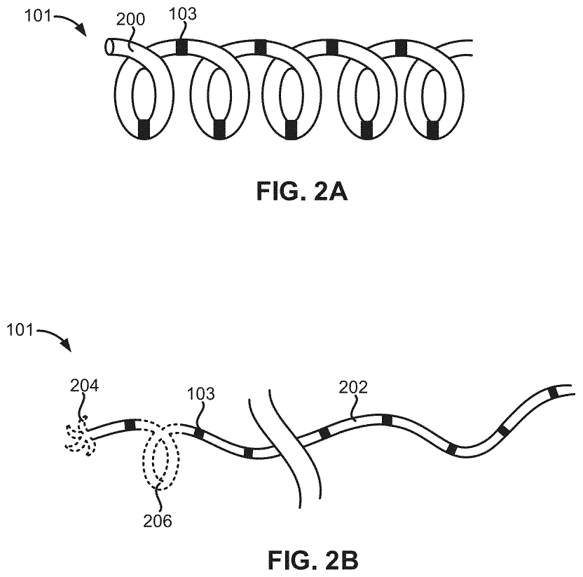

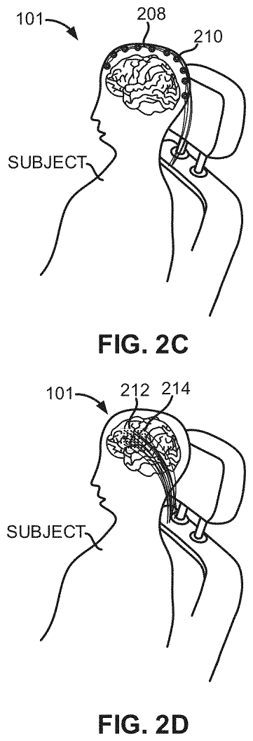

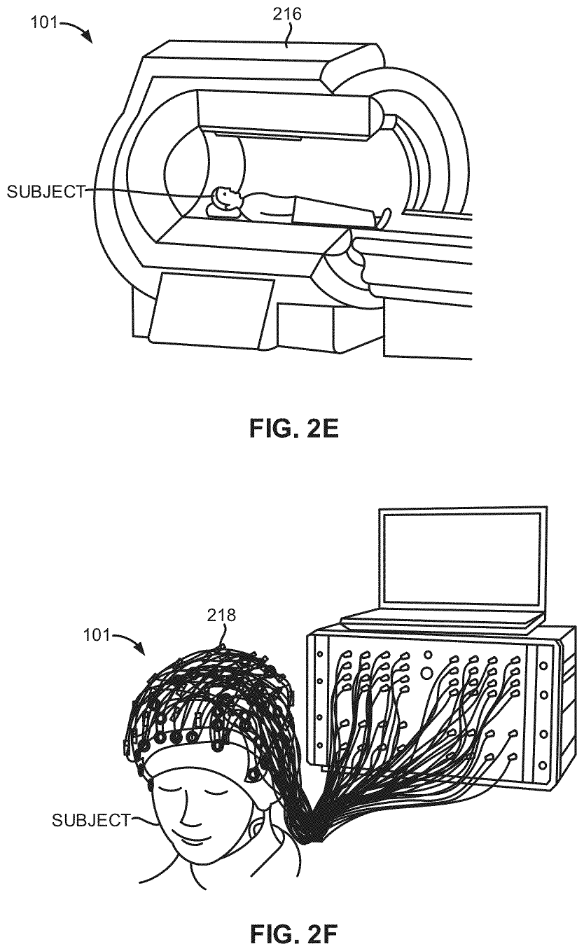

In other embodiments, the recording device 101 can be a non-invasive recording device 101 such as an electroencephalography (EEG) device (see, e.g., FIG. 2C), a functional magnetic resonance imaging (fMRI) device (see, e.g., FIG. 2E), or a functional near-infrared spectroscopy (fNIRS) device (see, e.g., FIG. 2F).

FIG. 1B illustrates that the stent-electrode array 102 can comprise a plurality of electrodes 103 affixed, secured, or otherwise coupled to an exterior portion or radially-outward portion of an expandable stent 105 or scaffold serving as an endovascular carrier for the electrode array. For example, the electrodes 103 can be arranged along filaments making up the walls or rings of the expandable stent 105.

In some embodiments, the filaments of the expandable stent 105 or scaffold can be made in part of a shape-memory alloy. For example, the filaments of the expandable stent 105 or scaffold can be made in part of Nitinol or Nitinol wire. The filaments of the expandable stent 105 or scaffold can also be made in part of stainless steel, gold, platinum, nickel, titanium, tungsten, aluminum, nickel-chromium alloy, gold-palladium-rhodium alloy, chromium-nickel-molybdenum alloy, iridium, rhodium, or a combination thereof.

In alternative embodiments, the filaments of the expandable stent 105 or scaffold can also be made in part of a shape memory polymer.

The electrodes 103 can be made in part of platinum, platinum black, gold, iridium, palladium, rhodium, or alloys or composites thereof (e.g., a gold-palladium-rhodium alloy or composite). In certain embodiments, the electrodes 103 can be made of a metal alloy or composite with a high charge injection capacity (e.g., a platinum-iridium alloy or composite).

The electrodes 103 can be shaped as circular disks having a disk diameter of between about 100 μm to 1.0 mm. In other embodiments, the electrodes 103 can have a disk diameter of between 1.0 mm and 1.5 mm. In other embodiments, the electrodes 103 can be cylindrical, spherical, cuff-shaped, ring-shaped, partially ring-shaped (e.g., C-shaped), or semi-cylindrical.

In other embodiments, the stent-electrode array 102 can be any of the stents, scaffolds, stent-electrodes, or stent-electrode arrays disclosed in U.S. Patent Pub. No. 2021/0365117; U.S. Patent Pub. No. 2021/0361950; U.S. Patent Pub. No. 2020/0363869; U.S. Patent Pub. No. 2020/0078195; U.S. Patent Pub. No. 2020/0016396; U.S. Patent Pub. No. 2019/0336748; U.S. Patent Pub. No. US 2014/0288667; U.S. Pat. Nos. 10,575,783; 10,485,968; 10,729,530, 10,512,555; U.S. Pat. App. No. 62/927,574 filed on Oct. 29, 2019; U.S. Pat. App. No. 62/932,906 filed on Nov. 8, 2019; U.S. Pat. App. No. 62/932,935 filed on Nov. 8, 2019; U.S. Pat. App. No. 62/935,901 filed on Nov. 15, 2019; U.S. Pat. App. No. 62/941,317 filed on Nov. 27, 2019; U.S. Pat. App. No. 62/950,629 filed on Dec. 19, 2019; U.S. Pat. App. No. 63/003,480 filed on Apr. 1, 2020; and U.S. Pat. App. No. 63/057,379 filed on Jul. 28, 2020, the contents of which are incorporated herein by reference in their entireties.

When the recording device 101 (e.g., the stent-electrode array 102) is implanted within a brain vessel 104 of the subject, each of the electrodes 103 of the recording device 101 can be configured to read or record the electrical activities of neurons within a vicinity of the electrode 103. The electrical activities of neurons are often recorded as rhythmic or repetitive patterns of activity that are also referred to as neural oscillations or brainwaves. Such neural oscillations or brainwaves can be further divided into bands by their frequency. For example, rhythmic neuronal activity between 14 Hz to 30 Hz is referred to as neuronal oscillations in a beta frequency range or beta-band.

When the recording device 101 (e.g., the stent-electrode array 102 of FIG. 1B) is implanted within a brain vessel 104 of the subject, the recording device 101 can record the neural oscillations of the subject, including any changes in such neural oscillations, over time in the beta-band (about 14 Hz to 30 Hz), alpha frequency range or alpha-band (about 7 Hz to 12 Hz), theta frequency range or theta-band (about 4 Hz to 7 Hz), gamma frequency range or gamma-band including a low frequency gamma-band (about 30 Hz to 70 Hz) and a high frequency gamma-band (about 70 Hz to 135 Hz), a delta frequency range or delta-band (about 0.1 Hz to 3 Hz), a mu frequency range or mu-band (about 7.5 Hz to 12.5 Hz), a sensorimotor rhythm (SMR) frequency range or SMR-band (about 12.5 Hz to 15.5 Hz), or a combination thereof. The recording device 101 can record changes in the power of such neural oscillations (e.g., as measured in decibels (dBs), micro-volts squared per Hz (μV2/Hz), average t-scores, average z-scores, etc.).

In some embodiments, the recording device 101 can be implanted within a cerebral or cortical vein or sinus of the subject. For example, the recording device 101 can be implanted within a superior sagittal sinus, an inferior sagittal sinus, a sigmoid sinus, a transverse sinus, a straight sinus, a superficial cerebral vein such as a vein of Labbe, a vein of Trolard, a Sylvian vein, a Rolandic vein, a deep cerebral vein such as a vein of Rosenthal, a vein of Galen, a superior thalamostriate vein, an inferior thalamostriate vein, or an internal cerebral vein, a central sulcal vein, a post-central sulcal vein, or a pre-central sulcal vein. In certain embodiments, the recording device 101 can be implanted within a vessel extending through a hippocampus or amygdala of the subject.

FIG. 1C illustrates that a communication conduit 108 (e.g., a lead wire) can connect the implantable recording device 101 (e.g., the stent-electrode array 102) with a telemetry unit 110 communicatively coupled to the computing device 106. Alternatively, a communication conduit 108 can connect the implantable recording device 101 directly with the computing device 106.

The communication conduit 108 can be a biocompatible lead wire or cable. When the recording device 101 is a stent-electrode array 102 deployed within a brain vessel (e.g., the superior sagittal sinus) 104 of the subject, the communication conduit 108 can extend through one or more brain vessels and out through a wall of a vein coupled to at least one of the brain vessels (e.g., the internal jugular vein) of the subject. The communication conduit 108 can then tunnel under the skin of the subject to a region of the subject (e.g., beneath the pectoralis major muscle) where the telemetry unit 110 is implanted.

FIG. 1D illustrates a close-up view of an embodiment of the telemetry unit 110. In some embodiments, the telemetry unit 110 can be configured to transmit signals received from the recording device 101 to the computing device 106 for processing and analysis. The telemetry unit 110 can also serve as a communication hub between the recording device 101 and the computing device 106. In certain embodiments, the computing device 106 can transmit commands or signals to the telemetry unit 110 to generate certain user outputs as part of a neurofeedback training regimen. Generating user outputs to train the subject to better control the BCI system 100 will be discussed in more detail in later sections.

In certain embodiments, the telemetry unit 110 can be an internal telemetry unit 110 implantable under the skin of the subject. For example, the telemetry unit 110 can be implanted within a pectoral region or within a subclavian space of the subject.

In other embodiments, the telemetry unit 110 can be an external telemetry unit 110 not implanted within the subject. In these embodiments, the communication conduit 108 can extend through the skin of the subject to connect to the telemetry unit 110. In additional embodiments, the telemetry unit 110 can comprise both an implantable portion and an external portion.

In some embodiments, the telemetry unit 110 can transmit data or signals to the computing device 106 or receive data or commands from the computing device 106 via a wired connection. In other embodiments, the telemetry unit 110 can transmit data or signals to the computing device 106 or receive data or commands from the computing device 106 via a wireless communication protocol such as Bluetooth™, Bluetooth Low Energy (BLE), ZigBee™, WiFi, or a combination thereof.

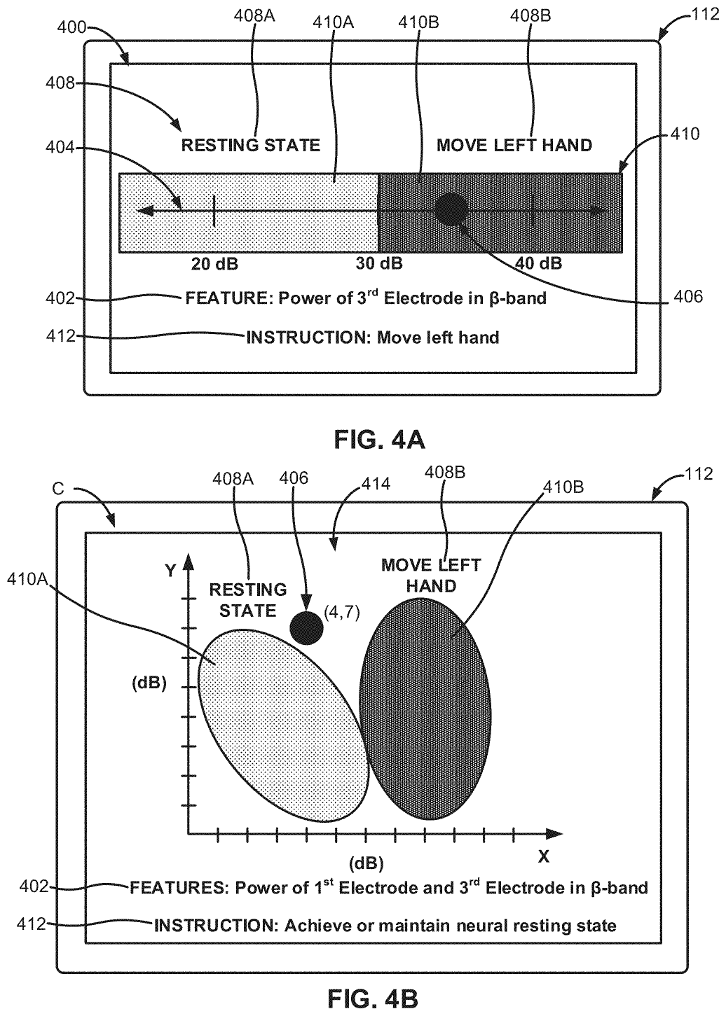

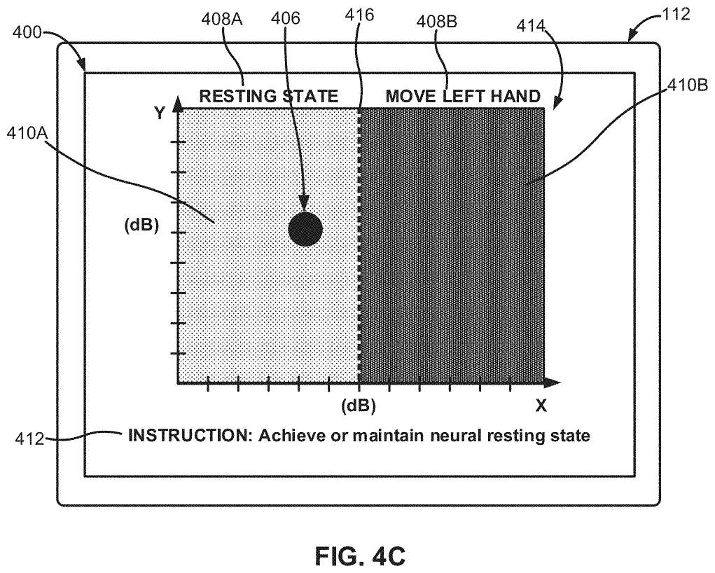

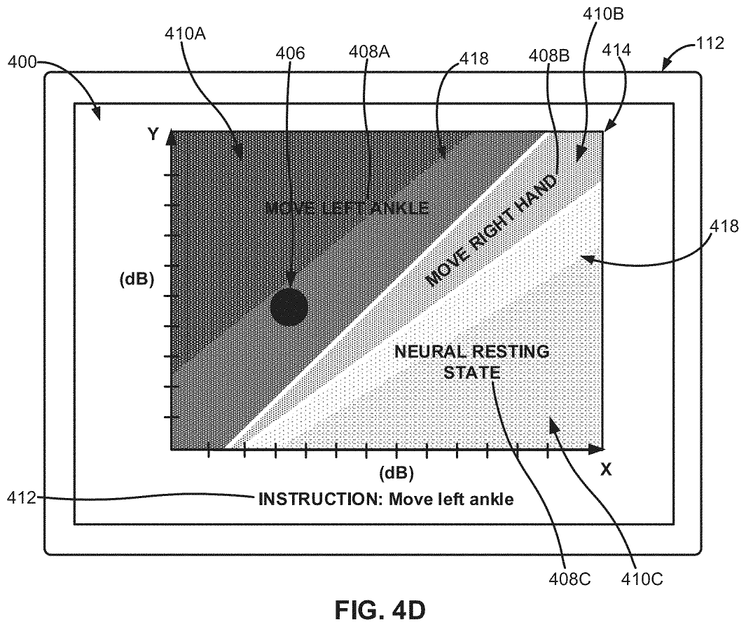

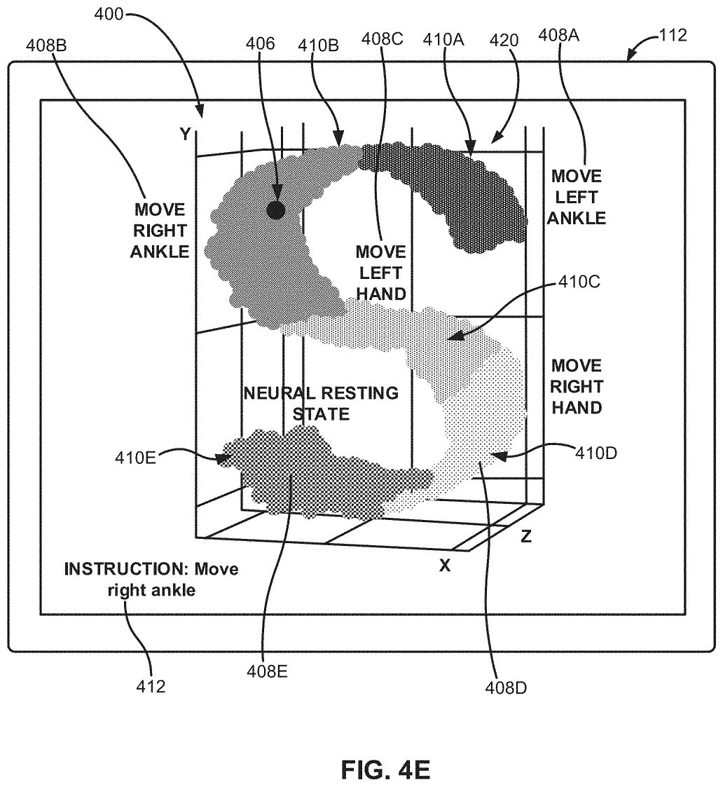

The computing device 106 can be programmed to convert brain activity recorded by the recording device 101 into predictions concerning an intention 408 (see FIGS. 4A-4E and 6-8) of the subject. As will be discussed in more detail in later sections, the intention 408 can be a thought conjured by the subject or an attempt made by the subject to move a body part of the subject (e.g., move a left hand or left ankle of the subject). Moreover, the intention 408 can also be a thought conjured by the subject or an attempt made by the subject to reach or maintain a neural rest state. In these cases, the intention 408 is not directly related to the subject focusing their attention or viewing certain graphics rendered on a display of a device such as the computing device 106.

The computing device 106 can convert brain activity recorded by the recording device 101 into predictions concerning the intention 408 of the subject by being trained to map or associate previously recorded brain activity to certain intentions 408. For example, the computing device 106 can be trained using training set data gathered from the subject as the subject repeatedly initiates, sustains, and terminates certain intentions 408. During these training sessions, the brain activity of the subject can be recorded by the recording device 101.

Once the computing device 106 is trained or calibrated using training set data gathered from the subject, the computing device 106 can control certain peripheral devices or software applications running on such peripheral devices based on the predicted intentions 408 of the subject. For example, the computing device 106 can be communicatively coupled to (i.e., in wired or wireless communication with) a peripheral device such as a personal electronic device, an IoT device, a mobility vehicle or a software application running on the peripheral device. The computing device 106 can transmit signals or commands to the peripheral device or the software application to control the operation or functionality of the peripheral device or the software application in response to the predicted intentions 408 of the subject. For example, the computing device 106 can instruct a mobility vehicle (e.g., a wheelchair) transporting the subject to move in a forward direction in response to the subject formulating or carrying out an intention 408 to move the subject's left hand.

However, as previously discussed, whether the subject is able to use the BCI system 100 to successfully control the peripheral device or software application depends on the ability of the subject to self-regulate their brain activity and to consistently produce brain activity calibrated to the intention 408. Therefore, neurofeedback training is needed to improve the subject's control over the BCI system 100 and, ultimately, improve the subject's control over one or more peripheral devices communicatively coupled to the BCI system 100 or software application running on such peripheral devices.

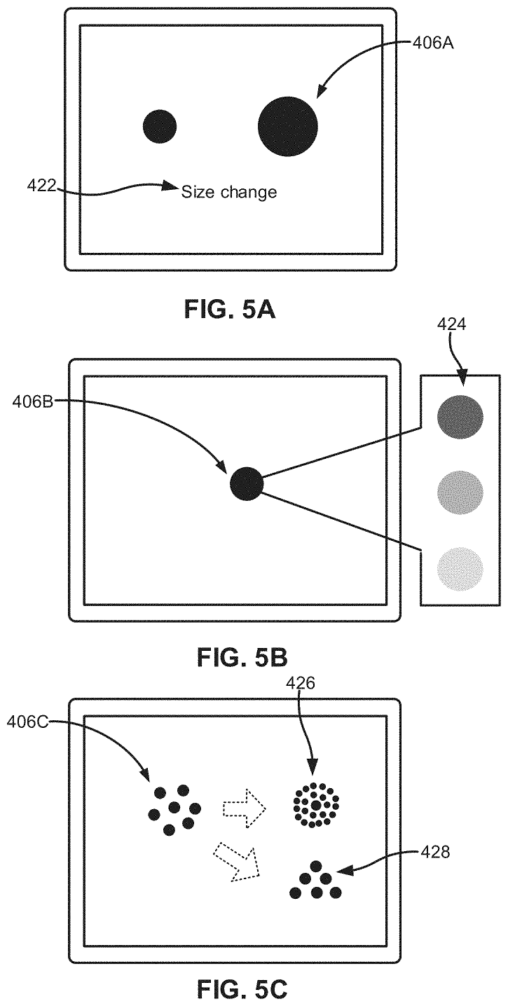

In some embodiments, a neurofeedback graphical user interface (GUI) 400 (see, e.g., FIGS. 4A-4E) can be displayed on a display 112 communicatively coupled to the computing device 106. The subject can view the neurofeedback GUI 400 on the display 112 while the subject is undergoing neurofeedback training. As will be discussed in more detail in later sections, a moveable graphic element 406 (see e.g., FIGS. 4A-4E and 5A-5C) can be shown on the neurofeedback GUI 400 representing a current brain activity of the subject recorded by the recording device 101. The neurofeedback GUI 400 and the graphic element 406 can be constructed in a way that reduces the complexity of the brain activity recorded by the recording device 101 into a form that is engaging and easy to comprehend by the subject. Moreover, the neurofeedback GUI 400 and the graphic element 406 can aid the subject in producing brain activity that aligns with a desired intention of the subject.

FIG. 2A illustrates another embodiment of the implantable recording device 101 as a coiled wire 200 comprising a plurality of electrodes 103. The coiled wire 200 can serve as the endovascular carrier for the electrodes 103 and can be used in vessels that are too small to accommodate the stent-electrode array 102.

The coiled wire 200 can be a biocompatible wire or microwire configured to wind itself into a coiled pattern or a substantially helical pattern. The electrodes 103 can be arranged such that the electrodes 103 are scattered along a length of the coiled wire 200. More specifically, the electrodes 103 can be affixed, secured, or otherwise coupled to distinct points along a length of the coiled wire 200.

The electrodes 103 can be separated from one another such that no two electrodes 103 are within a predetermined separation distance (e.g., at least 10 μm, at least 100 μm, or at least 1.0 mm) from one another. In some embodiments, the wire 200 can be configured to automatically wind itself into a coiled configuration (e.g., helical pattern) when the wire 200 is deployed out of a delivery catheter. For example, the coiled wire 200 can automatically attain its coiled configuration via shape memory when the delivery catheter or sheath is retracted. The coiled configuration or shape can be a preset or shape memory shape of the wire 200 prior to the wire 200 being introduced into a delivery catheter. The preset or pre-trained shape can be made to be larger than the diameter of the anticipated deployment or implantation vessel to enable the radial force exerted by the coils to secure or position the coiled wire 200 in place within the deployment or implantation vessel.

The wire 200 can be made in part of a shape-memory alloy, a shape-memory polymer, or a combination thereof. For example, wire 200 can be made in part of Nitinol (e.g., Nitinol wire). The wire 200 can also be made in part of stainless steel, gold, platinum, nickel, titanium, tungsten, aluminum, nickel-chromium alloy, gold-palladium-rhodium alloy, chromium-nickel-molybdenum alloy, iridium, rhodium, or a combination thereof.

FIG. 2B illustrates yet another embodiment of the implantable recording device 101 as an anchored wire 202 comprising a plurality of electrodes 103. The anchored wire 202 can serve as the endovascular carrier for the electrodes 103 and can be used in vessels that are too small to accommodate either the coiled wire 200 or the stent-electrode array 102.

The anchored wire 202 can comprise a biocompatible wire or microwire attached or otherwise coupled to an anchor or another type of endovascular securement mechanism. FIG. 2B illustrates that the anchored wire 202 can comprise a barbed anchor 204, a radially-expandable anchor 206, or a combination thereof (both the barbed anchor 204 and the radially-expandable anchor 206 are shown in broken or phantom lines in FIG. 2B). In some embodiments, the barbed anchor 204 can be positioned at a distal end of the anchored wire 202. In other embodiments, the barbed anchor 204 can be positioned along one or more sides of the wire or microwire. The barbs of the barbed anchor 204 can secure or moor the anchored wire 202 to an implantation site within the subject. The radially-expandable anchor 206 can be a segment of the wire or microwire shaped as a coil or loop. The coil or loop can be sized to allow the coil or loop to conform to a vessel lumen and to expand against a lumen wall to secure the anchored wire 202 to an implantation site within the vessel. For example, the coil or loop can be sized to be larger than the diameter of the anticipated deployment or implantation vessel to enable the radial force exerted by the coil or loop to secure or position the anchored wire 202 in place within the deployment or implantation vessel.

The electrodes 103 of the anchored wire 202 can be scattered along a length of the anchored wire 202. More specifically, the electrodes 103 can be affixed, secured, or otherwise coupled to distinct points along a length of the anchored wire 202. The electrodes 103 can be separated from one another such that no two electrodes 103 are within a predetermined separation distance (e.g., at least 10 μm, at least 100 μm, or at least 1.0 mm) from one another. Although FIG. 2B illustrates the anchored wire 202 having only one barbed anchor 204 and one radially-expandable anchor 206, it is contemplated by this disclosure that the anchored wire 202 can comprise a plurality of barbed anchors 204 and/or radially-expandable anchors 206.

FIG. 2C illustrates that in another embodiment of the system 100, the recording device 101 can be a non-invasive device such as an electroencephalogram (EEG) device 208. The EEG device 208 can be a head-mounted EEG apparatus. For example, the EEG device 208 can be an EEG cap or an EEG-visor configured to be worn by the subject. The EEG device 208 can comprise a plurality of non-invasive electrodes 210 configured to be in contact with the scalp of the subject.

The brain activity detected by the EEG device 208 can be neural oscillations or brainwaves of the subject, similar to those recorded by the implantable recording device 101. For example, the EEG device 208 can record neural oscillations, including any changes in such neural oscillations, over time in the beta-band (about 14 Hz to 30 Hz), alpha frequency range or alpha-band (about 7 Hz to 12 Hz), theta frequency range or theta-band (about 4 Hz to 7 Hz), gamma frequency range or gamma-band including a low frequency gamma-band (about 30 Hz to 70 Hz) and a high frequency gamma-band (about 70 Hz to 135 Hz), a delta frequency range or delta-band (about 0.1 Hz to 3 Hz), a mu frequency range or mu-band (about 7.5 Hz to 12.5 Hz), a sensorimotor rhythm (SMR) frequency range or SMR-band (about 12.5 Hz to 15.5 Hz), or a combination thereof. The EEG device 208 can record changes in the power of such neural oscillations (e.g., as measured in decibels (dBs), micro-volts squared per Hz (μV2/Hz), average t-scores, average z-scores, etc.).

FIG. 2D illustrates that in yet another embodiment of the system 100, the recording device 101 can be an electrocorticography (ECoG) device 212 (also referred to as an intracranial EEG device). The ECoG device 10 can be a flexible or stretchable electrode-mesh or one or more electrode patches implanted or placed on a surface of the brain of the subject. The electrode-mesh or electrode patch can comprise a plurality of electrodes 214 arranged on the mesh or patch, respectively.

The brain activity detected by the ECoG device 212 can be neural oscillations or brainwaves of the subject, similar to those recorded by the stent-electrode array 102. For example, the ECoG device 212 can record neural oscillations, including any changes in such neural oscillations, over time in the beta-band (about 14 Hz to 30 Hz), alpha frequency range or alpha-band (about 7 Hz to 12 Hz), theta frequency range or theta-band (about 4 Hz to 7 Hz), gamma frequency range or gamma-band including a low frequency gamma-band (about 30 Hz to 70 Hz) and a high frequency gamma-band (about 70 Hz to 135 Hz), a delta frequency range or delta-band (about 0.1 Hz to 3 Hz), a mu frequency range or mu-band (about 7.5 Hz to 12.5 Hz), a sensorimotor rhythm (SMR) frequency range or SMR-band (about 12.5 Hz to 15.5 Hz), or a combination thereof. The ECoG device 212 can record changes in the power of such neural oscillations (e.g., as measured in decibels (dBs), micro-volts squared per Hz (μV2/Hz), average t-scores, average z-scores, etc.).

FIG. 2E illustrates that in another embodiment of the system 100, the recording device 101 can be a functional magnetic resonance imaging (fMRI) machine 216. The fMRI machine 216 can detect changes in blood flow and blood-oxygen levels within the brain of the subject as the subject engages in neural activity as part of the neurofeedback training. Such changes in blood flow and blood-oxygen levels are the indirect consequence of the subject's neural activity.

In some embodiments, the fMRI machine 216 can measure the brain activity of the subject using blood-oxygen-level dependent (BOLD) contrast imaging. For example, the brain activity of the subject can be expressed as changes in the BOLD signal. In other embodiments, the fMRI machine 216 can measure the brain activity of the subject using arterial spin labeling (ASL) rather than BOLD contrast imaging.

FIG. 2F illustrates that in another embodiment of the system 100, the recording device 101 can be a functional near-infrared spectroscopy (fNIRS) device 218. The fNIRS device 218 can use near-infrared light (NIR) to measure hemodynamic activity in the brain of the subject as the subject engages in neural activity as part of the neurofeedback training. For example, the fNIRS device 218 can comprise a fNIRS cap configured to be worn on the head of the subject. The fNIRS device 218 can comprise a plurality of NIR light sources and detectors (called optodes). The fNIRS device 218 can measure the hemodynamic activity by measuring changes in oxy-hemoglobin concentrations (HbO) and deoxy-hemoglobin (HbR) concentrations in the cerebral cortex.

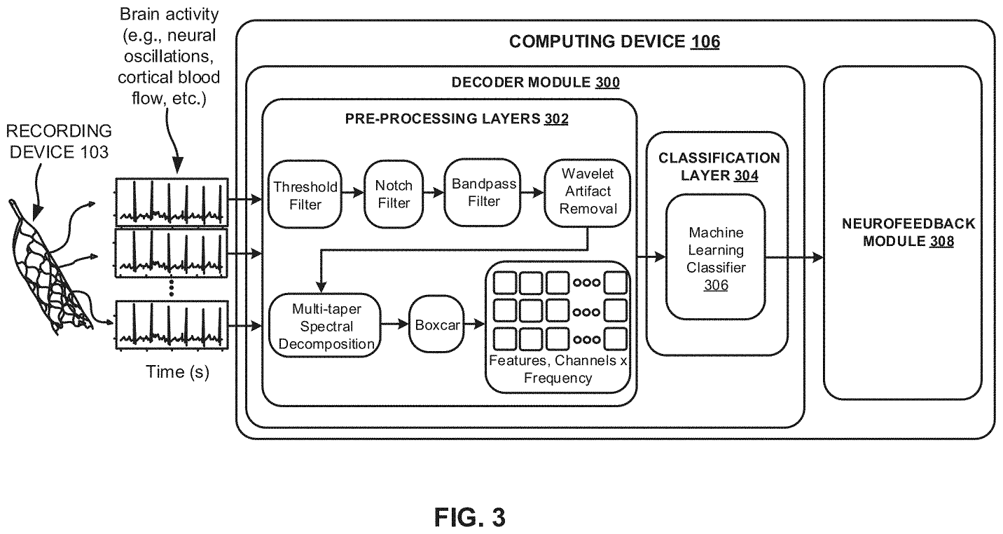

FIG. 3 illustrates certain software layers or modules running on the computing device 106 of the BCI system 100. As shown in FIG. 1A, the computing device 106 can be a standalone computing device separate from the telemetry unit 110 and the recording device 101. For example, one or more processors of the computing device 106 can be programmed to execute software instructions making up the various software layers or modules. In this example embodiment, the telemetry unit 110

In other embodiments not shown in the figures but contemplated by this disclosure, any references to the computing device 106 can also refer to a control unit or controller embedded within the telemetry unit 110. In further embodiments contemplated by this disclosure, any references to the computing device 106 can also refer to a computing device or control unit/controller that is part of the recording device 101 (for example, when the recording device 101 is an fMRI machine or an fNIRS device).

As shown in FIG. 3, the computing device 106 can comprise at least a decoder module 300 and a neurofeedback module 308. The decoder module 300 can further comprise one or more pre-processing layers 302 and a classification layer 304.

The pre-processing layer 302 can comprise a plurality of software filters or filtering modules configured to filter and smooth out the raw signals obtained from the recording device 101. For example, when the recording device 101 is an endovascular recording device configured to be implanted within a brain vessel of the subject(e.g., the stent-electrode array 102), the brain activity of the subject can be monitored using the various electrodes 103 of the recording device 101. As a more specific example, the brain activity of the subject can be sampled every 100 ms such that 100 ms “chunks” or bins of the raw neural signals recorded can be passed to the pre-processing layer 302 for processing and smoothing.

The pre-processing layer 302 can first apply a (1) threshold filter to filter out the raw signals using certain thresholds. The pre-processing layer 302 can then apply a (2) notch filter to perform, for example, 50 Hz notch filtering and also apply a (3) bandpass filter to perform, for example, 4-30 Hz Butterworth bandpass filtering. The pre-processing layer 302 can then apply a (4) wavelet artifact removal filter to perform wavelet-based artifact rejection, a (5) multi-taper spectral decomposition filter to perform multi-taper spectral decomposition, and a (6) boxcar smoothing filter to perform temporal boxcar smoothing. The filtered data can then be fed to the classification layer 304 of the decoder module 300.

The classification layer 304 can comprise one or more machine learning algorithms or classifiers 306 to classify the resulting data segments or bins into an intention 408 (see FIGS. 4A-4E and 6-8) of the subject. In some embodiments, the machine learning algorithm or classifier 306 can be a supervised learning model such as a support vector machine (SVM). In other embodiments, the machine learning algorithm or classifier can be a Gaussian mixture model classifier, a Naïve Bayes classifier, or another type of machine learning classifier.

The classification layer 304 can be trained or calibrated to classify or make predictions concerning the intention 408 of the subject based on previously recorded brain activity. For example, the classification layer 304 can predict the subject's intentions 408 several times per second. The classification layer 304 can be trained using training data collected from the subject.

In some embodiments, the training phase can involve the subject repeatedly initiating, sustaining, and terminating certain thoughts or attempting certain actions while the subject's brain activity is recorded by the recording device 101. For example, one such training session can involve the subject repeatedly resting for 5 seconds followed by attempting to move their left hand for 5 seconds. The subject's brain activity during this training session can be recorded and the recorded brain activity can be mapped to the subject's intentions 408 to rest and move their left hand, respectively.

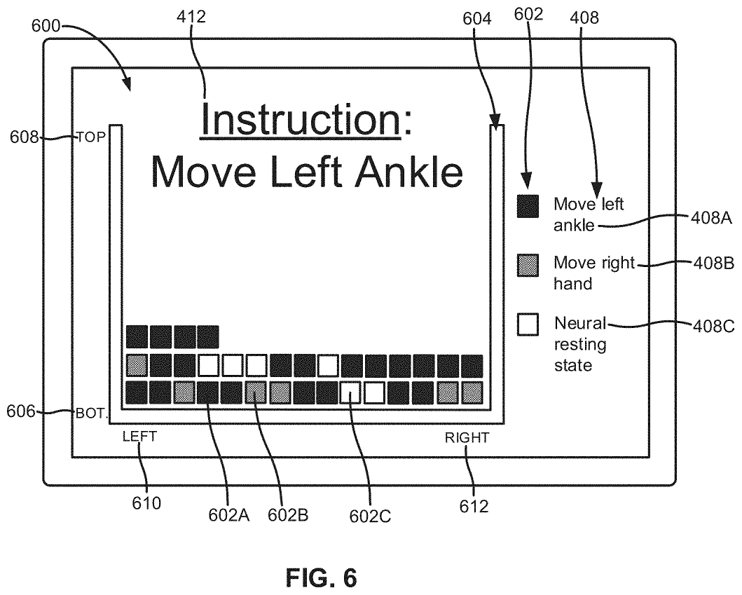

As shown in FIG. 3, the classification layer 304 can feed the predicted intention 408 of the subject to the neurofeedback module 308. In some embodiments, the neurofeedback module 308 can be configured to construct certain neurofeedback GUIs (see, e.g., FIGS. 4A-4E and 6) to be displayed to the subject via a display 112 communicatively coupled to the computing device 106 to aid the subject in producing or recreating brain activity that aligns with a desired intention of the subject. In other embodiments, the neurofeedback module 308 can also transmit commands or signals to a user output device (e.g., a speaker or a tactile feedback component) communicatively coupled to the computing device 106 to generate a user output (e.g., sounds or tactile feedback) to aid the subject in producing brain activity that aligns with a desired intention of the subject. The neurofeedback module 308 will be discussed in more detail in the following sections.

FIG. 4A illustrates one embodiment of a neurofeedback GUI 400 constructed by the neurofeedback module 308 (see FIG. 3) of the computing device 106. The neurofeedback GUI 400 can be displayed to the subject via a display 112 communicatively coupled to the computing device 106. The neurofeedback GUI 400 can assist the subject in consistently producing brain activity that aligns with a desired intention of the subject.

One technical problem faced by the applicant is how to design a neurofeedback training program that would be effective in training the subject without overwhelming the subject with all of the data collected by the recording device 101. For example, when the recording device 101 is an implantable recording device such as the stent-electrode array 102 of FIG. 1B, the recording device 101 can comprise typically between 16 to 64 electrodes 103. For each electrode, there are many frequency bands that contain useful information regarding the subject's intentions 408. This combination of electrodes 103 and frequency bands gives rise to many (e.g., >100) potential markers of brain activity (termed “features”) that can be useful to show the subject. Moreover, frequency values can also be calculated across varying time windows that can give rise to an additional dimension of possible features. One technical solution discovered and developed by the applicant is to reduce the numerous markers of brain activity recorded by the recording device 101 (the numerous features) into one representative marker (or one feature) of brain activity. By doing so, the multivariate data collected from the recording device 101 is reduced to univariate data that can then be used to construct the neurofeedback GUI 400 to be displayed to the subject as part of the neurofeedback training.

FIG. 4A illustrates that one representative feature 402 can be rendered via the neurofeedback GUI 400 as a number line 404. A graphic element 406 can be rendered as movable along the number line 404. The graphic element 406 can represent a current brain activity of the subject recorded by the recording device 101.

For example, the representative feature 402 can be a power of one of the electrodes 103 (e.g., the third electrode) of the recording device 101 in a beta-band frequency (e.g., between 14 Hz and 30 Hz). The decoder module 300 can determine, based on past training sessions with the subject, that a power level of around 20 dB in this particular electrode 103 and this frequency band corresponds to the subject achieving or maintaining a neural resting state and a power level of around 40 dB in this electrode 103 and this frequency band corresponds to an attempt by the subject (or a thought conjured by the subject) to move their left hand. The subject's attempt to achieve either the neural resting state or to move the subject's left hand can be considered an intention 408 of the subject.

In addition to the number line 404, additional graphics can be presented via the neurofeedback GUI 400 that reveal, to the subject, how brain activity is classified by the machine learning classifier 306 of the decoder module 300. The possible outcomes of this classification (referred to in machine learning terminology as a “class”) are the intentions 408 of the subject.

For example, FIG. 4A shows that the number line 404 used to convey the representative feature 402 (e.g., power level of one of the electrodes of recording device 101 in the beta-band frequency) can be presented over a plurality of graphic portions 410. For example, the plurality of graphic portions 410 can comprise a first graphic portion 410A representing a first intention 408A and a second graphic portion 410B representing a second intention 408B. As a more specific example, the first intention 408A can be an intention of the subject to achieve or maintain a neural rest state and the second intention 408B can be an intention of the subject to move their left hand. The first graphic portion 410A and the second graphic portion 410B can visually represent classification outcomes that can be outputted by the machine learning classifier 306 based on the subject's past brain activity recorded by the recording device 101.

In these embodiments, neither the first intention 408A nor the second intention 408B is directly related to the subject focusing their attention or viewing the graphic portions 410. For example, the intentions 408 (either the first intention 408A or the second intention 408B) can be related to a thought conjured by the subject or an attempt made by the subject to move a body part of the subject or to maintain a neural rest state.

In some embodiments, the graphic portions 410, including the first graphic portion 410A and the second graphic portion 410B, can be different-colored background shading used to demarcate different segments of the number line 404. For example, the first graphic portion 410A can be a first rectangular box behind part of the number line 404 colored grey while the second graphic portion 410B can be a second rectangular box contiguous with the first rectangular box but colored blue.

In other embodiments not shown in FIG. 4A, the first graphic portion 410A can refer to a segment of the number line 404 colored a first color (e.g., grey) and the second graphic portion 410B can refer to another segment of the number line 404 colored a second color different from the first color (e.g., blue).

The boundaries of the graphic portions 410, including the boundaries of the first graphic portion 410A and the boundaries of the second graphic portion 410B, can be established based on the previously recorded brain activity of the subject during the training phase. For example, the first intention 408A of the subject (e.g., achieving or maintaining a neural rest state) can be calibrated to a recorded power level in the beta-band frequency of one of the electrodes 103 at around 20 dB. The first graphic portion 410A symbolizing this predictive tendency of the classification layer 304 can encompass the segment of the number line 404 in the vicinity of the 20 dB power level along with segments of the number line 404 slightly below the 20 dB power level and up to the 30 dB power level. Also, in this example, the second intention 408B of the subject (attempting a left hand movement) can be calibrated to a recorded power level in the beta-band frequency of one of the electrodes 103 at around 40 dB. The second graphic portion 410B symbolizing this predictive tendency of the classification layer 304 can encompass the segment of the number line 404 in the vicinity of the 40 dB power level along with segments of the number line 404 slightly above the 40 dB power level and down to the 30 dB power level. The 30 dB power level can be selected as the intention boundary (or decision boundary) given its position as the midpoint between the 20 dB and 40 dB power levels. Alternatively, an intention boundary can be selected based on how often the power level detected exceeds or fails to meet this boundary level.

As shown in FIG. 4A, in some embodiments, the entire number line 404 can appear within the graphic portions 410. In other embodiments, only part of or a segment of the number line 404 can appear within the graphic portions 410. Moreover, although only two intentions 408 and two graphic portions 410 are shown in the neurofeedback GUI 400 of FIG. 4A, it is contemplated by this disclosure that instances of the neurofeedback GUI 400 can be constructed that comprise three or more intentions 408 and graphic portions 410. In these embodiments, multiple intention boundaries (or decision boundaries) can be calculated or selected based on the subject's previously recorded brain activity.

FIG. 4A also illustrates that the neurofeedback GUI 400 can also comprise a graphic element 406 representing a current brain activity of the subject recorded by the recording device 101. In the example embodiment shown in FIG. 4A, the graphic element 406 can represent the brain activity of the subject as it pertains to the one representative feature 402 (e.g., the power level of one of the electrodes in the beta-band frequency).

In some embodiments, the graphic element 406 can be a dot (e.g., a black dot) movable along the feature number line 404. In other embodiments not shown in FIG. 4A but contemplated by this disclosure, the graphic element 406 can be another type of graphical icon or symbol movable along the number line 404. The graphic element 406 can move along the number line 404 and between the two graphic portions 410 as the brain activity of the subject changes during a neurofeedback training session. The purpose of the graphic element 406 is to motivate and train the subject to consistently produce or generate brain activity that aligns with a desired intention of the subject.

For example, the neurofeedback GUI 400 can display an instruction 412 (e.g., a text instruction) to the subject to move their left hand. The subject can then attempt to produce or activate brain activity to satisfy the instruction 412. For example, there are distinct ways for a human subject to focus the mind to achieve certain intentions such as moving the subject's left hand. These can involve repetitive pulses of thoughts concerning the movement or short repetitive attempts to undertake the movement. Such short repetitive thoughts or attempts can be distinguished from a long sustained thought or attempt to undertake the movement. The subject can try out different mental activation strategies or focusing techniques to see which works best to move the graphic element 406 in the desired direction or maintains the graphic element 406 in the desired graphic portion 410. In this manner, the graphic element 406 can provide real-time or near-real-time feedback to the subject concerning how successful they are in controlling the BCI system 100 on command.

As previously discussed, since each of the calibrated intentions 408 of the subject can be associated with a command to control a peripheral device communicatively coupled to the computing device 106, the subject can improve their control over such peripheral devices (or software running on such devices) by undergoing neurofeedback training in the manner disclosed herein.

In some embodiments, the neurofeedback GUI 400, or components thereof, can be written using the Java™ programming language, the Python™ programming language, the C/C++ programming language, the JavaScript programming language, the Ruby™ programming language, the C# programming language, or a combination thereof.

FIG. 4B illustrates another embodiment of the neurofeedback GUI 400. The neurofeedback GUI 400 can be constructed by the neurofeedback module 308 (see FIG. 3) and displayed to the subject via the display 112 as part of a neurofeedback training session.

The neurofeedback GUI 400 is an example of a user interface where multivariate data concerning the brain activity of the subject (for example, multiple features or markers of brain activity recorded by the recording device 101) are rendered as graphics displayed in a two-dimensional (2D) space and shown to the subject as part of the subject's neurofeedback training. For example, as previously discussed, when the recording device 101 is an implantable recording device (such as the stent-electrode array 102 of FIG. 1B), the recording device 101 can comprise typically between 16 to 64 electrodes 103. For each of these electrodes 103, there are many neural oscillation frequency bands that contain useful information regarding the subject's intentions 408.

This combination of electrodes 103 and frequency bands can give rise to many (e.g., >100) potential markers of brain activity or features that can be useful to show the subject. Moreover, frequency values can also be calculated across varying time windows that can give rise to an additional dimension of possible features. The main technical problem faced by the applicant is how to design a neurofeedback training program that would be effective in training the subject without overwhelming the subject with too much data. One technical solution discovered and developed by the applicant is to reduce the numerous markers of brain activity recorded by the recording device 101 to a more manageable subset of markers or features. In this manner, multivariate data collected from the recording device 101 is presented to the subject but the complexity of such multivariate data is reduced to an extent that is easy to comprehend for the subject.

The neurofeedback module 308 can use certain dimensionality reduction functions to reduce multivariate data or high-dimensional features to a lower dimensional subspace (e.g., a 2D subspace). In some embodiments, the dimensionality reduction function can be a projection matrix.

For example, the recording device 101 can be an implantable recording device (e.g., the stent-electrode array 102) capable of recording a “high-dimensional” set of features including the power of at least four electrodes 103 (e.g., electrodes 1, 2, 3, and 4) of the recording device 101 in a beta-band frequency (e.g., between 14 Hz and 30 Hz). A particular instance of these features can be represented by the vector x below:

$$x = [ \begin{matrix}4 \\ - 2 \\ 7 \\ 3\end{matrix} ] = [ \begin{matrix}Power in electrode 1 = 4 dB \\ Power in electrode 2 = - 2 dB \\ Power in electrode 3 = 7 dB \\ Power in electrode 4 = 3 dB\end{matrix} ]$$

The above four-dimensional feature vector (x) can be projected into two-dimensions by left multiplying x with a 2×4 projection matrix. One example of this projection matrix (A) is presented below that would display the electrode 1 power on an x-axis of a two-dimensional (2D) graph 414 (see, e.g., FIG. 4B) and display the electrode 3 power on a y-axis of the same 2D graph 414:

$$A = [ \begin{matrix}1 & 0 & 0 & 0 \\ 0 & 0 & 1 & 0\end{matrix} ]$$

Multiplying the projection matrix (A) with the four-dimensional (4D) feature vector (x) yields the below 2D vector (z):

$$z = A \cdot x = [ \begin{matrix}1 & 0 & 0 & 0 \\ 0 & 0 & 1 & 0\end{matrix} ] \cdot [ \begin{matrix}4 \\ - 2 \\ 7 \\ 3\end{matrix} ] = [ \begin{matrix}4 \\ 7\end{matrix} ]$$

The resulting 2D vector (z) can be the representative features 402 shown to the subject through the neurofeedback GUI 400. In the example above, these can include the electrode 1 power and the electrode 3 power. These extracted representative features 402 can then be shown on a 2D graph as an individual point.

In some embodiments, principal component analysis (PCA) can be used to calculate a projection matrix that can capture most of the variance in the higher-dimensional data. Calculating the projection matrix using PCA will be discussed in more detail in later sections.

FIG. 4B illustrates an embodiment of the neurofeedback GUI 400 where a dimensionality reduction function (e.g., a projection matrix) is used to reduce previously recorded high-dimensional brain activity data and currently recorded high-dimensional brain activity data to a lower-dimensional set of data that can then be represented through the 2D graph 414. This embodiment of the neurofeedback GUI 400 also comprises a plurality of graphic portions 410 including at least a first graphic portion 410A and a second graphic portion 410B.

The first graphic portion 410A can be a first area on the 2D graph 414 representing a first intention 408A of the subject calibrated to certain previously recorded brain activity of the subject. The first area can be comprised of points representing 2D feature vectors obtained from reducing higher-dimensional feature vectors (i.e., greater than 2D) using a projection matrix. The second graphic portion 410B can be a second area on the 2D graph 414 representing a second intention 408B of the subject calibrated to certain previously recorded brain activity of the subject. The second area can be comprised of points representing 2D feature vectors obtained from reducing higher-dimensional feature vectors (i.e., greater than 2D) using the same projection matrix. The first graphic portion 410A and the second graphic portion 410B can visually represent classification outcomes that can be outputted by the machine learning classifier 306 based on the subject's past brain activity recorded by the recording device 101.

As shown in the example embodiment of FIG. 4B, the first graphic portion 410A can be a visual representation of lower-dimensional feature vectors of the subject associated with a neural resting state. Moreover, the second graphic portion 410B can be a visual representation of lower-dimensional feature vectors of the subject associated with a left-hand movement. The lower-dimensional feature vectors can be reduced from higher-dimensional feature vectors using one or more dimensionality reduction functions.

The higher-dimensional feature vectors can be obtained from numerous training sessions conducted with the subject where the machine learning classifier 306 is trained using training set data gathered from the subject as the subject repeatedly initiates, sustains, and terminates certain intentions 408. During these training sessions, the brain activity of the subject can be recorded by the recording device 101.

For example, training set data in the form of higher-dimensional feature vectors can be obtained from training sessions where the subject repeatedly initiates, sustains, and terminates actions or thoughts related to the first intention 408A (e.g., achieving or maintaining a neural resting state) or the second intention 408B (e.g., moving the left hand of the subject) while the brain activity of the subject is being recorded by the recording device 101.

Although the example above shows the higher-dimensional feature vector as a 4D vector, it is contemplated by this disclosure and it should be understood by one of ordinary skill in the art that the higher-dimensional feature vector can comprise more than four features. Moreover, although FIG. 4B shows the features used to generate the neurofeedback GUI 400 as the powers of two electrodes in the beta-band frequency, it is contemplated by this disclosure and it should be understood by one of ordinary skill in the art that the features can be any number of markers of brain activity that can be representative of an intention 408 of the subject or that can be useful to show the subject.

FIG. 4B also illustrates that this embodiment of the neurofeedback GUI 400 can also comprise a graphic element 406. The neurofeedback module 308 can construct the graphic element 406 to appear on the 2D graph 414 based on a current brain activity of the subject recorded by the recording device 101.

In some embodiments, the graphic element 406 can be a colored dot moveable within the 2D graph 414. The graphic element 406 can also be shown either within the first graphic portion 410A or the second graphic portion 410B. The graphic element 406 can also move between the first graphic portion 410A and the second graphic portion 410B based on the current brain activity of the subject recorded by the recording device 101. In these embodiments, the graphic element 406 is moveable (controlled by the brain activity of the subject) while the graphic portions (e.g., the first graphic portion 410A and the second graphic portion 410B) are static.

In some embodiments, the first graphic portion 410A can be presented or displayed in a first color and the second graphic portion 410B can be presented or displayed in a second color. The second color can be different from the first color so as to allow the subject to more easily distinguish between the first graphic portion 410A and the second graphic portion 410B. In certain embodiments, the graphic portions 410 (e.g., the first graphic portion 410A and the second graphic portion 410B) can be arranged and colored according to the distribution of the training data set.

As shown in FIG. 4B, the neurofeedback GUI 400 can also display an instruction 412 to the subject to formulate or carry out either the first intention 408A or the second intention 408B as part of the neurofeedback training. The subject can then attempt to produce or activate brain activity to satisfy the instruction 412.

For example, the instruction 412 can instruct the subject to achieve or maintain a neural resting state. The subject can utilize the neurofeedback GUI 400 to try out different mental activation strategies or focusing techniques to see which works best to move the graphic element 406 into the graphic portion 410 associated with achieving or maintaining a neural rest state (e.g., the first graphic portion 410A) or to keep the graphic element 406 within the graphic portion 410. In this manner, the graphic element 406 can provide real-time or near-real-time feedback to the subject concerning how successful they are in self-regulating their brain activity. This, in turn, can provide the subject feedback on how successful they are in controlling the BCI system 100 on command.

As previously discussed, since each of the calibrated intentions 408 of the subject can be associated with a command to control a peripheral device communicatively coupled to the computing device 106, the subject can improve their control over such peripheral devices (or software running on such devices) by undergoing neurofeedback training in the manner disclosed herein.

In some embodiments, the neurofeedback GUI 400, or components thereof, can be written using the Java™ programming language, the Python™ programming language, the C/C++ programming language, the JavaScript programming language, the Ruby™ programming language, the C# programming language, or a combination thereof.

As previously discussed, the graphic portions 410 (e.g., the first graphic portion 410A and the second graphic portion 410B) and the graphic element 406 of the neurofeedback GUI 400 of FIG. 4B are visual representations of recorded brain activity data that has been reduced using a dimensionality reduction function. Projection matrices are one type of dimensionality reduction function that can reduce higher dimensional features to a lower dimensional subspace. One technical problem faced by the applicants is that different projection matrices will display, but also hide, different aspects of the higher dimensional data. Therefore, it is important to use a projection matrix that can yield lower dimensional features that can capture most of the variance in the higher dimensional features. One technical solution discovered and developed by the applicants is to use principal component analysis (PCA) to calculate a projection matrix that can yield lower dimensional features that can capture the variance in the higher dimensional features.

PCA is a dimensionality reduction technique that finds different “lenses” through which to view higher dimensional brain activity feature data according to the amount of variation. Disclosed herein is a method of calculating a projection matrix using PCA. The method can comprise: (i) calculating a first principal component, (ii) calculating a second principal component, and (iii) combining the two principal components into a 2×M projection matrix, where M is the original number of features.

The first principal component of the higher dimensional features, referred to as X below, can be calculated using Equation 1 below:

ΣX(p₁)=λ₁(p₁) (Equation 1)

where ΣX is the sample covariance matrix of X, λ₁ is the largest eigenvalue and p₁ is the eigenvector corresponding to the largest eigenvalue.

The second principal component of X can be calculated using Equation 2 below:

ΣX(p₂)=λ₂ (p₂) (Equation 2)

where ΣX is the sample covariance matrix of X, λ₂ is the second largest eigenvalue and p₂ is the eigenvector corresponding to the second largest eigenvalue.

The sample covariance matrix, ΣX, can be calculated using Equation 3 below:

$$\sum X = \frac{1}{N - 1} X {X}^{T} \qquad ( Equation 3 )$$

The first principal component refers to a first projection vector (a “lens”) that captures the most amount of variability in the higher-dimensional brain activity feature data. The second principal component refers to a second projection vector (another “lens”) that captures the second most amount of variability.

The first projection vector and the second projection vector can be combined, using Equation 4 below, to make a 2×M projection matrix, referred to as f_{2D} below:

$${f}_{2 D =} [ \begin{matrix}{p}_{1}^{T} \\ {p}_{2}^{T}\end{matrix} ] \qquad ( Equation 4 )$$

The projection matrix, f_{2D}, can then be used to convert the M-dimensional features into two-dimensional features, Z, using Equation 5 below:

$$Z = {f}_{2 D} ( X ) = [ \begin{matrix}{p}_{1}^{T} \\ {p}_{2}^{T}\end{matrix} ] \cdot X \qquad ( Equation 5 )$$

where the first and second rows of Z are displayed on the x- and y-axes of the 2D graph 414 of the neurofeedback GUI 400.

The projection matrix, f_{2D}, above is a linear dimensionality reduction function that can show the higher-dimensional brain activity data according to two directions with the most variability.

The method disclosed above can be stored as machine-executable instructions stored on a non-transitory computer-readable medium, such as a non-transitory computer-readable medium (e.g., the memory) of the computing device 106. In some embodiments, the one or more processors of the computing device 106 can be programmed to execute the method steps disclosed above in order to calculate the projection matrix using PCA.

Although the method steps above discuss using two principal components (e.g., the first principal component and the second principal component), it is contemplated by this disclosure that more than two principal components can also be used. Other methods to linearly reduce the feature space can include independent component analysis or common spatial patterns.

FIG. 4C illustrates yet another embodiment of the neurofeedback GUI 400. The neurofeedback GUI 400 can be constructed by the neurofeedback module 308 (see, e.g., FIG. 3) and displayed to the subject via the display 112 as part of a neurofeedback training session. Similar to the embodiment of the neurofeedback GUI 400 shown in FIG. 4B, this embodiment of the neurofeedback GUI 400 can also comprise a 2D graph 414 comprising at least a first graphic portion 410A, a second graphic portion 410B, and a moveable graphic element 406.

However, in this embodiment of the neurofeedback GUI 400, the multivariate data or high-dimensional features recorded by the recording device 101 can be reduced into (or projected onto) two dimensions using a hyperplane of a linear classifier or linear classification algorithm. Reducing the high-dimensional features in this manner can allow the graphic portions 410 (e.g., the 406 first graphic portion 410A and the second graphic portion 410B) to occupy the entire 2D graph 414 such that every pixel of the background of the 2D graph 414 is colored according to class predictions (in the form of the subject's intentions 408) made by the machine learning classifier 306 (see, e.g., FIG. 3).

In some embodiments, the linear classification algorithm can be a linear discriminant analysis (LDA) classifier. In other embodiments, the linear classification algorithm can be a support vector machine (SVM) or another type of machine learning linear classifier.

As shown in FIG. 4C, the 2D graph 414 can comprise an x-axis and a y-axis. The x-axis projection can be defined according to the decision boundary of the linear classification algorithm. The decision boundary of the linear classification algorithm is also known as a hyperplane 416. In some embodiments, the y-axis can be projected according to another method such as with a principal component. For example, the y-axis can be projected using the largest principal component that is orthogonal to the hyperplane 416. Assigning the y-axis as the largest principal component means that the data will be seen through the lens of where it is varying the most, along the y-axis. This has the effect of separating out the two classes (e.g., the two intentions 408) on either side of the hyperplane 416. This means that the area to the left side of the hyperplane 416, shown in FIG. 4C as the first graphic portion 410A, corresponds to one class or one intention 408 (e.g., the first intention 408A) and the area to the right side of the hyperplane 416, shown in FIG. 4C as the second graphic portion 410B, corresponds to another class or another intention 408 (e.g., the second intention 408B). The hyperplane 416 is indicated in FIG. 4C using the black broken line.

In an alternative embodiment, the y-axis can be projected using another linear classifier hyperplane.

The neurofeedback module 308 can construct the graphic element 406 to appear on the 2D graph 414 based on a current brain activity of the subject recorded by the recording device 101. In some embodiments, the graphic element 406 can be a colored dot moveable within the 2D graph 414. The graphic element 406 can also be shown either within the first graphic portion 410A or the second graphic portion 410B.

The graphic element 406 can move between the first graphic portion 410A and the second graphic portion 410B based on the current brain activity of the subject recorded by the recording device 101. In these embodiments, the graphic element 406 is moveable (controlled by the brain activity of the subject) while the graphic portions (e.g., the first graphic portion 410A and the second graphic portion 410B) are static.

In some embodiments, the first graphic portion 410A can be presented or displayed in a first color and the second graphic portion 410B can be presented or displayed in a second color. The second color can be different from the first color so as to allow the subject to more easily distinguish between the first graphic portion 410A and the second graphic portion 410B.

As shown in FIG. 4C, an instruction 412 can be displayed to the subject to formulate or carry out either the first intention 408A or the second intention 408B as part of the subject's neurofeedback training. The subject can then attempt to produce or activate brain activity to satisfy the instruction 412. For example, an instruction can be displayed to the subject to achieve or maintain a neural resting state. The subject can utilize the neurofeedback GUI 400 to try out different mental activation strategies or focusing techniques to see which works best to move the graphic element 406 into the graphic portion 410 associated with achieving or maintaining the neural rest state (e.g., the first graphic portion 410A) or to keep the graphic element 406 within the graphic portion 410. In this manner, the graphic element 406 can provide real-time or near-real-time feedback to the subject concerning how successful they are in self-regulating their brain activity. This, in turn, can provide the subject feedback on how successful they are in controlling the BCI system 100 on command.

In some embodiments, the neurofeedback GUI 400, or components thereof, can be written using the Java™ programming language, the Python™ programming language, the C/C++ programming language, the JavaScript programming language, the Ruby™ programming language, the C# programming language, or a combination thereof.

FIG. 4D illustrates an additional embodiment of the neurofeedback GUI 400. The neurofeedback GUI 400 can be constructed by the neurofeedback module 308 (see, e.g., FIG. 3) and displayed to the subject via the display 112 as part of a neurofeedback training session. This embodiment of the neurofeedback GUI 400 can also comprise a 2D graph 414 comprising a plurality of graphic portions 410 including at least a first graphic portion 410A, a second graphic portion 410B, a third graphic portion 410C and a moveable graphic element 406.

In this embodiment of the neurofeedback GUI 400, a dimensionality reduction function (e.g., a projection matrix) can be used to reduce multivariate data or high-dimensional features to a lower dimensional subspace (e.g., the 2D subspace or the 2D graph 414 shown in FIG. 4D). For example, principal component analysis (PCA) can be used to calculate a projection matrix that can capture most of the variance in the higher-dimensional data. However, some of the higher-dimensional data can be lost through the dimensionality reduction procedure. For example, if PCA is used to calculate the projection matrix, the first principal component and the second principal component can capture 95% of the variance of the data. This leaves 5% of the data that will be lost through the dimensionality reduction procedure. Therefore, another method of generating the neurofeedback GUI 400 is to project each pixel in the 2D plane back to the original feature space (the higher-dimensional feature space) using an inverse transform of a linear forward projection and then classify this resulting point with the machine learning classifier 306 (see, e.g., FIG. 3).

Continuing the example from above, the two-dimensional features, Z, calculated from Equation 5 above can be projected back to the original feature space using f_{2D}⁻¹. Moreover, the predicted label for each element of the feature map can be determined using f_{transform}(Z) The feature map can then be colored according to the predicted labels accordingly.

The caveat to this method is that some pixels in the 2D plane may have multiple valid classifier predictions due to the areas of the data hidden by the dimensionality reduction procedure. Thus, it is beneficial to indicate some level of uncertainty in the back-projected predictions. As shown in FIG. 4D, this can be done using color gradients 418 according to the probability of the classifier's predictions.

Once again, each point on the 2D graph 414 or 2D feature map of FIG. 4D can correspond to a point in the original high-dimensional or multivariate feature space where the mapping is done via PCA decomposition of previously-recorded brain activity data obtained from the subject in previous training sessions. The graphic portions 410 can then be constructed by coloring each of the mapped points based on a class prediction for the associated high-dimensional feature.

Each of the graphic portions 410 (any of the first graphic portion 410A, the second graphic portion 410B, or the third graphic portion 410C) can represent an intention of the subject calibrated to certain previously-recorded brain activity of the subject.

Moreover, the first graphic portion 410A can be presented or displayed in a first color, the second graphic portion 410B can be presented or displayed in a second color, and the third graphic portion 410C can be presented or displayed in a third color. The first color, the second color, and the third color can all be different so as to allow the subject to more easily distinguish between the first graphic portion 410A, the second graphic portion 410B, and the third graphic portion 410C.

The neurofeedback module 308 can construct the graphic element 406 to appear on the 2D graph 414 based on a current brain activity of the subject recorded by the recording device 101. In some embodiments, the graphic element 406 can be a colored dot moveable within the 2D graph 414. The graphic element 406 can also be shown within any of the first graphic portion 410A, the second graphic portion 410B, or the third graphic portion 410C.

The graphic element 406 can move between the graphic portions 410 (e.g., the first graphic portion 410A, the second graphic portion 410B, and the third graphic portion 410C) based on the current brain activity of the subject recorded by the recording device 101. In these embodiments, the graphic element 406 is moveable (controlled by the brain activity of the subject) while the graphic portions (e.g., the first graphic portion 410A, the second graphic portion 410B, and the third graphic portion 410C) are static.

In the example embodiment shown in FIG. 4D, the first graphic portion 410A can represent a first intention 408A of the subject. The first intention 408A can be an attempt by the subject (or a thought conjured by the subject) to move their left ankle. The second graphic portion 410B can represent a second intention 408B of the subject. The second intention 408B can be an attempt by the subject (or a thought conjured by the subject) to move their right hand. The third graphic portion 410C can represent a third intention 408C of the subject. The third intention 408C can be an attempt by the subject (or a thought conjured by the subject) to achieve or maintain a neural resting state.

As previously discussed, since there can be some degree of uncertainty in the back-projected predictions made by the classifier, a color gradient can be used to color the graphic portions 410 according to the probability of the classifier's predictions. For example, a darker color can indicate a high degree of certainty in the class (e.g., intention) prediction and a lighter color can indicate a lower degree of certainty in the class prediction.

As shown in FIG. 4D, an instruction 412 can be displayed to the subject to formulate or carry out an intention 408 (any of the first intention 408A, the second intention 408B, or the third intention 408C) as part of the subject's neurofeedback training. The subject can then attempt to produce or activate brain activity to satisfy the instruction 412. For example, an instruction can be displayed to the subject to move the subject's left ankle. The subject can utilize the neurofeedback GUI 400 to try out different mental activation strategies or focusing techniques to see which works best to move the graphic element 406 into the graphic portion 410 associated with moving the subject's left ankle (e.g., the first graphic portion 410A) or to keep the graphic element 406 within the graphic portion 410. In this manner, the graphic element 406 can provide real-time or near-real-time feedback to the subject concerning how successful they are in self-regulating their brain activity. This, in turn, can provide the subject feedback on how successful they are in controlling the BCI system 100 on command.

In some embodiments, the neurofeedback GUI 400, or components thereof, can be written using the Java™ programming language, the Python™ programming language, the C/C++ programming language, the JavaScript programming language, the Ruby™ programming language, the C# programming language, or a combination thereof.

FIG. 4E illustrates a further embodiment of the neurofeedback GUI 400. The neurofeedback GUI 400 can be constructed by the neurofeedback module 308 (see, e.g., FIG. 3) and displayed to the subject via the display 112 as part of a neurofeedback training session. This embodiment of the neurofeedback GUI 400 can also comprise a three-dimensional (3D) graph 420 comprising a plurality of graphic portions 410 including at least a first graphic portion 410A, a second graphic portion 410B, a third graphic portion 410C, a fourth graphic portion 410D, a fifth graphic portion 410E, and a moveable graphic element 406.

In this embodiment of the neurofeedback GUI 400, a non-linear dimensionality reduction algorithm or technique can be used to reduce multivariate data or high-dimensional features to a lower dimensional subspace (e.g., the 3D subspace or the 3D graph 420 shown in FIG. 4E). Examples of non-linear dimensionality reduction algorithms or techniques can comprise: a (1) locally-linear embedding (LLE) algorithm or technique; a (2) Hessian LLE algorithm or technique; a (3) modified LLE algorithm or technique; a (4) local tangent space alignment (LTSA) algorithm or technique; an (5) isomap algorithm or technique; a (6) multidimensional scaling (MDS) algorithm or technique; a (7) spectral embedding algorithm or technique; or a (8) t-distributed stochastic neighbor embedding (t-SNE) algorithm or technique.

Each point on the 3D graph 420 or 3D feature map of FIG. 4E can correspond to a point in the original high-dimensional or multivariate feature space where the mapping is done via non-linear dimensionality reduction of previously-recorded brain activity data obtained from the subject in previous training sessions.

Each of the graphic portions 410 (any of the first graphic portion 410A, the second graphic portion 410B, the third graphic portion 410C, the fourth graphic portion 410D, the fifth graphic portion 410E) can represent an intention of the subject calibrated to certain previously-recorded brain activity of the subject.

Moreover, the first graphic portion 410A can be presented or displayed in a first color, the second graphic portion 410B can be presented or displayed in a second color, the third graphic portion 410C can be presented or displayed in a third color, the fourth graphic portion 410D can be presented or displayed in a fourth color, and the fifth graphic portion 410E can be presented or displayed in a fifth color. The first color, the second color, the third color, the fourth color, and the fifth color can all be different so as to allow the subject to more easily distinguish between the first graphic portion 410A, the second graphic portion 410B, the third graphic portion 410C, the fourth graphic portion 410D, and the fifth graphic portion 410E.

The neurofeedback module 308 can construct the graphic element 406 to appear on the 3D graph 420 based on a current brain activity of the subject recorded by the recording device 101. In some embodiments, the graphic element 406 can be a colored dot moveable within the 3D graph 420. The graphic element 406 can also be shown within any of the first graphic portion 410A, the second graphic portion 410B, the third graphic portion 410C, the fourth graphic portion 410D, or the fifth graphic portion 410E.

The graphic element 406 can move between the graphic portions 410 (e.g., the first graphic portion 410A, the second graphic portion 410B, the third graphic portion 410C, the fourth graphic portion 410D, and the fifth graphic portion 410E) based on the current brain activity of the subject recorded by the recording device 101. In these embodiments, the graphic element 406 is moveable (controlled by the brain activity of the subject) while the graphic portions (e.g., the first graphic portion 410A, the second graphic portion 410B, the third graphic portion 410C, the fourth graphic portion 410D, and the fifth graphic portion 410E) are static.

In the example embodiment shown in FIG. 4E, the first graphic portion 410A can represent a first intention 408A of the subject. The first intention 408A can be an attempt by the subject (or a thought conjured by the subject) to move their left ankle. The second graphic portion 410B can represent a second intention 408B of the subject. The second intention 408B can be an attempt by the subject (or a thought conjured by the subject) to move their right ankle. The third graphic portion 410C can represent a third intention 408C of the subject. The third intention 408C can be an attempt by the subject (or a thought conjured by the subject) to move their left hand. The fourth graphic portion 410D can represent a fourth intention 408D of the subject. The fourth intention 408D can be an attempt by the subject (or a thought conjured by the subject) to move their right hand. The fifth graphic portion 410E can represent a fifth intention 408E of the subject. The fifth intention 408E can be an attempt by the subject (or a thought conjured by the subject) to achieve or maintain a neural resting state.

A color gradient can also be used to color the graphic portions 410 according to the probability of the classifier's predictions. For example, a darker color can indicate a high degree of certainty in the class (e.g., intention) prediction and a lighter color can indicate a lower degree of certainty in the class prediction.

As shown in FIG. 4E, an instruction 412 can be displayed to the subject to formulate or carry out an intention 408 (any of the first intention 408A, the second intention 408B, the third intention 408C, the fourth intention 408D, or the fifth intention 408E) as part of the subject's neurofeedback training. The subject can then attempt to produce or activate brain activity to satisfy the instruction 412. For example, an instruction can be displayed to the subject to move the subject's right ankle. The subject can utilize the neurofeedback GUI 400 to try out different mental activation strategies or focusing techniques to see which works best to move the graphic element 406 into the graphic portion 410 associated with moving the subject's right ankle (e.g., the second graphic portion 410B) or to keep the graphic element 406 within the graphic portion 410. In this manner, the graphic element 406 can provide real-time or near-real-time feedback to the subject concerning how successful they are in self-regulating their brain activity. This, in turn, can provide the subject feedback on how successful they are in controlling the BCI system 100 on command.

In some embodiments, the neurofeedback GUI 400, or components thereof, can be written using the Java™ programming language, the Python™ programming language, the C/C++ programming language, the JavaScript programming language, the Ruby™ programming language, the C# programming language, or a combination thereof.

FIG. 5A illustrates another embodiment of the graphic element 406 (indicated as graphic element 406A in FIG. 5A) used to depict a current brain activity of the subject recorded by the recording device 101. In this embodiment, a size 422 of the graphic element 406A can change or be dynamically adjusted to represent a certainty level or probability that the graphic element 406A belongs in a particular graphic portion 410. For example, when the graphic element 406A is a circular dot, a diameter of the circular dot can increase if the machine learning classifier 306 (see FIG. 3) is more certain that the graphic element 406A belongs in a particular graphic portion 410 and the diameter of the circular dot can decrease if the machine learning classifier 306 is less certain that the graphic element 406A belongs in a certain graphic portion 410. The graphic element 406A shown in FIG. 5A can be any of the graphic elements 406 shown in or described with respect to FIGS. 4A, 4B, 4C, 4D, or 4E.

In some embodiments, the certainty value can be determined by obtaining a probability of the class prediction from the classification layer 304. For example, if a linear discriminant analysis (LDA) classifier is used to color the graphic portions 410, then the same LDA classifier can be used to obtain probability values for each class prediction of the current brain activity of the subject and use such probability values to determine an appropriate size 422 for the graphic element 406A.

As previously discussed, the graphic element 406A can move between different graphic portions 410 to provide real-time or near-real-time feedback to the subject concerning how successful the subject is in self-regulating their brain activity. The subject can try out different mental activation strategies or focusing techniques to see which works best to move the graphic element 406A into a desired graphic portion 410 or to keep the graphic element 406 within the desired graphic portion 410.

FIG. 5B illustrates another embodiment of the graphic element 406 (indicated as graphic element 406B in FIG. 5B) used to depict a current brain activity of the subject recorded by the recording device 101. In this embodiment, a color or color intensity 424 of the graphic element 406B can change or be dynamically adjusted to represent a certainty level or probability that the graphic element 406B belongs in a particular graphic portion 410. For example, when the graphic element 406B is a circular dot, a hue of the circular dot or a color saturation of the circular dot can be adjusted. As a more specific example, the color saturation or color intensity of the graphic element 406B can be increased or the graphic element 406B can be darkened if the machine learning classifier 306 (see FIG. 3) is more certain that the graphic element 406B belongs in a particular graphic portion 410. Also, in this example, the color saturation or color intensity of the graphic element 406B can be decreased or the graphic element 406B can be lightened if the machine learning classifier 306 is less certain that the graphic element 406B belongs in a particular graphic portion 410.

In some embodiments, the certainty level of the class prediction (e.g., the prediction concerning the intention 408 of the subject) made by the machine learning classifier 306 can be calculated as a percentage value between 0% and 100%. In these embodiments, thresholds can be set by the neurofeedback module 308 relating to the certainty level such that the graphic element 406B is of a particular hue or color saturation level when the certainty level outputted by the machine learning classifier 306 reaches a particular threshold.

The graphic element 406B shown in FIG. 5B can be any of the graphic elements 406 shown in or described with respect to FIGS. 4A, 4B, 4C, 4D, or 4E.

FIG. 5C illustrates yet another embodiment of the graphic element 406 (indicated as a graphic element swarm 406C in FIG. 5C) used to depict a current brain activity of the subject recorded by the recording device 101. In this embodiment, a singular graphic element 406 can be replaced by a collection of smaller graphic elements or a graphic element swarm 406C. A density 426 or shape 428 of the graphic element swarm 406C can change or be dynamically adjusted to represent a certainty level or probability that the graphic element swarm 406C belongs in a particular graphic portion 410. For example, when the graphic element swarm 406C is a circular-shaped graphic composed of a collection of smaller circular-shaped graphics, the density of the graphic element swarm 406C can be increased or the smaller circular-shaped graphics can be more closely packed together if the machine learning classifier 306 (see FIG. 3) is more certain that the graphic element swarm 406C belongs in a particular graphic portion 410. Also, in this example, the density of the graphic element swarm 406C can be decreased or the graphic element swarm 406C can be composed of fewer constituent graphic elements if the machine learning classifier 306 is less certain that the graphic element swarm 406C belongs in a particular graphic portion 410.

In additional embodiments, the shape 428 of the graphic element swarm 406C can also be dynamically adjusted or changed based on a certainty level of the class prediction (e.g., the prediction concerning the intention 408 of the subject) made by the machine learning classifier 306. For example, the certainty level can be calculated as a percentage value between 0% and 100%. In this example, the graphic element swarm 406C can form a first shape (e.g., a circular shape) if the certainty level of the class prediction made by the machine learning classifier 306 is above a predetermined threshold level (e.g., a certainty level of above 90%). Also, in this example, the graphic element swarm 406C can form a second shape (e.g., a triangular shape, see FIG. 5C) if the certainty level of the class prediction is below the predetermined threshold level.

In a further embodiment, the shape 428 of the graphic element swarm 406C can also represent the second most probable class prediction (or intention 408). For example, the graphic element swarm 406C can come together to form a triangular shape to represent a prediction made by the machine learning classifier 306 that the subject intends to move the subject's hands. In this example, the machine learning classifier 306 can make a low probability prediction that the current brain activity of the subject recorded by the recording device 101 matches an intention 408 by the subject to move the ankles of the subject. Also, in this example, the second most probable prediction made by the machine learning classifier 306 is an intention 408 by the subject to move the hands of the subject. In this case, the graphic element swarm 406C can be shown as a less dense swarm of smaller graphic elements shaped as a triangle appearing over a graphic portion 410 representing an intention 408 by the subject to move the subject's hands. In this manner, the shape 428 of the graphic element swarm 406C can be used to convey additional information besides the positioning or location of the graphic element swarm 406C within the various graphic portions 410.

The graphic element swarm 406C shown in FIG. 5C can be any of the graphic elements 406 shown in or described with respect to FIG. 4A, 4B, 4C, 4D, or 4E.

FIG. 6 illustrates a further embodiment of a neurofeedback GUI 600. The neurofeedback GUI 600 can be constructed by the neurofeedback module 308 (see FIG. 3) and displayed to the subject via the display 112 as part of a neurofeedback training session.

The neurofeedback GUI 600 is an example of a user interface where multivariate data concerning the brain activity of the subject (for example, multiple features or markers of brain activity recorded by the recording device 101) are rendered as graphics presented to the subject through the display 112 to aid the subject in producing brain activity that align with a desired intention 408 of the subject.

In some embodiments, one or more processors of the computing device 106 (see FIG. 1A) can be programmed to execute or carry out instructions stored as part of the neurofeedback module 308 (see FIG. 3) to associate graphic elements 602 with certain intentions 408 of the subject. More specifically, the one or more processors of the computing device 106 can be programmed to associate at least a first graphic element 602A with a first intention 408A of the subject calibrated to certain previously recorded brain activity of the subject and associate at least a second graphic element 602B with a second intention 408B of the subject calibrated to certain previously recorded brain activity of the subject.

As shown in FIG. 6, the one or more processors of the computing device 106 can also be programmed to associate a third graphic element 602C with a third intention 408C of the subject calibrated to certain previously recorded brain activity of the subject. Although three graphic elements 602 are depicted in the example neurofeedback GUI 600 shown in FIG. 6, it is contemplated by this disclosure that four or more types of graphic elements 602 can also be used to provide neurofeedback training to the subject.

In this embodiment, the display 112 can be configured to display instances of the graphic elements 602 in temporal succession based on the current brain activity of the subject recorded by the recording device 101. For example, the display 112 can be configured to display instances of any of the first graphic element 602A, the second graphic element 602B, or the third graphic element 602C in temporal succession based on the current brain activity of the subject recorded by the recording device 101.

For purposes of the present disclosure, “in temporal succession” means that each of the graphic elements 602 are displayed in succession (i.e., one at a time) to the subject over time. Each of the graphic elements 602 represents a prediction made by the machine learning classifier 306 (see FIG. 3) concerning an intention 408 of the subject based on the current brain activity recorded by the recording device 101.

The brain activity of the subject can be recorded as multivariate data by the recording device 101. The multivariate data obtained from the recording device 101 can be fed to the classification layer 304 and the machine learning classifier 306 can be trained to make predictions concerning an intention 408 of the subject based on the recorded data.

In some embodiments, the machine learning algorithm or classifier 306 can be a supervised learning model such as a support vector machine (SVM). In other embodiments, the machine learning algorithm or classifier can be a Gaussian mixture model classifier, a Naïve Bayes classifier, or another type of machine learning classifier.

The classification layer 304 can be trained or calibrated to classify or make predictions concerning the intention 408 of the subject based on previously recorded brain activity. The classification layer 304 can be trained using training data collected from the subject. For example, the machine learning classifier 306 can be trained using training set data gathered from the subject as the subject repeatedly initiates, sustains, and terminates certain intentions 408 while the brain activity of the subject is recorded by the recording device 101.

As shown in FIG. 6, the neurofeedback GUI 600 can also display an instruction 412 to the subject to formulate or carry out an intention 408 such as moving a left ankle of the subject. The intention 408 can be one of the intentions 408 (any of the first intention 408A, the second intention 408B, or the third intention 408C) associated with the graphic elements 602. The subject can then produce brain activity that aligns with or achieves the intention 408 indicated as part of the instruction 412. This intention 408 can also be referred to as a desired intention of the subject.

In the example shown in FIG. 6, the subject can produce brain activity that aligns with or achieves the intention 408 of moving the left ankle of the subject. The brain activity produced or generated by the subject can then be displayed to the subject through the neurofeedback GUI 600 in the form of the graphic elements 602. The subject can try out different mental activation strategies or focusing techniques to see which works best to cause more of the desired graphic elements 602 (e.g., the first graphic elements 602A) to appear. The goal for the subject is to adjust their neural activation strategies or focusing techniques until more of their desired graphic elements 602 appear on the screen

In this manner, the graphic elements 602 can provide real-time or near-real-time feedback to the subject concerning how successful they are in self-regulating their brain activity. This, in turn, can provide the subject feedback on how successful they are in controlling the BCI system 100 on command. As previously discussed, since each of the calibrated intentions 408 of the subject can be associated with a command to control a peripheral device communicatively coupled to the computing device 106, the subject can improve their control over such peripheral devices (or software running on such devices) by undergoing neurofeedback training in the manner disclosed herein.

In some embodiments, the neurofeedback GUI 600 can show the graphic elements 602 (any of the first graphic element 602A, the second graphic element 602B, or the third graphic element 602C) as filling up a container graphic 604. The container graphic 604 can be a graphic representation of a cross-section of a container with an open top (e.g., a graphic of a container having a container base and two container walls).

In certain embodiments, the graphic elements 602 can be rendered as filling up the container graphic 604 from bottom 606 to top 608. In these and other embodiments, the graphic elements 602 can also be rendered as filling up the container graphic 604 from left 610 to right 612.

Each of the graphic elements 602 can represent a prediction made by the machine learning classifier 306 based on a recording made by the recording device 101 of the brain activity of the subject at a particular point in time. For each, a new graphic element 602 can appear in the container graphic 604 every second, every half second, every 1/10 of a second, every 1/100 of a second, or every millisecond. How often or quickly the graphic elements 602 appear in the container graphic 604 can be adjusted based on the reaction time of the subject or other factors.

The graphic elements 602 can be visually distinct from one another so that the subject can easily distinguish between the various graphic elements 602. For example, the graphic elements 602 can be different colored squares appearing in a white container graphic 604. As a more specific example, the first graphic element 602A can be a blue-colored square, the second graphic element 602B can be a red-colored square, and the third graphic element 602C can be a white-colored square.

In other embodiments not shown in the figures, the graphic elements 602 can be shaped differently from one another and/or sized differently from one another. In certain embodiments, each of the graphic elements 602 can have its own unique color, shape, or size.

As shown in the example embodiment in FIG. 6, the graphic elements 602 can be presented as discrete elements separated by spaces. In other embodiments not shown in the figures but contemplated by this disclosure, similar graphic elements 602 can be connected to one another or presented as contiguous with one another such that no spaces separate adjacent graphic elements 602.

In the example embodiment shown in FIG. 6, previously rendered graphic elements 602 (i.e., graphic elements 602 representing past points in time) are kept on the screen or visually persist or remain on the screen and are not visually removed or erased after they first appear. In this manner, the subject is able to see the amount of time they were able to successfully regulate or control their brain activity over an entire training period.

The objective of the neurofeedback GUI 600 is to allow the subject to see the internal predictive behavior of the machine learning classifier 306 at a high temporal resolution (i.e., in real-time or near real-time) so the subject can learn from these training sessions and improve control over their own brain activity.

In some embodiments, the neurofeedback GUI 600, or components thereof, can be written using the Java™ programming language, the Python™ programming language, the C/C++ programming language, the JavaScript programming language, the Ruby™ programming language, the C# programming language, or a combination thereof.

Referring back to the FIG. 1A, in some embodiments, a subject may have difficulty distinguishing between visual elements on a graphical interface (e.g., when the subject is visually impaired). In these embodiments, it is useful to be able to provide alternate modes of feedback to the subject as part of the neurofeedback training sessions.

Moreover, alternate modes of feedback can also be provided to subjects that can perceive visual elements on a graphical interface. Such alternate modes of feedback can be provided in addition to (rather than in lieu of) the visual feedback provided as part of the neurofeedback GUIs disclosed herein (e.g., the neurofeedback GUI 400 or the neurofeedback GUI 600).

In one embodiment, an auditory component 114 (see, e.g., FIG. 1A), such as a speaker, can be included as part of the BCI system 100. For example, the auditory component 114 can be a built-in speaker of the display 112. Also, for example, the auditory component 114 can be a stand-alone speaker communicatively coupled to the computing device 106. In a further example, the auditory component 114 can be a speaker integrated into the telemetry unit 110 (see FIG. 1A).

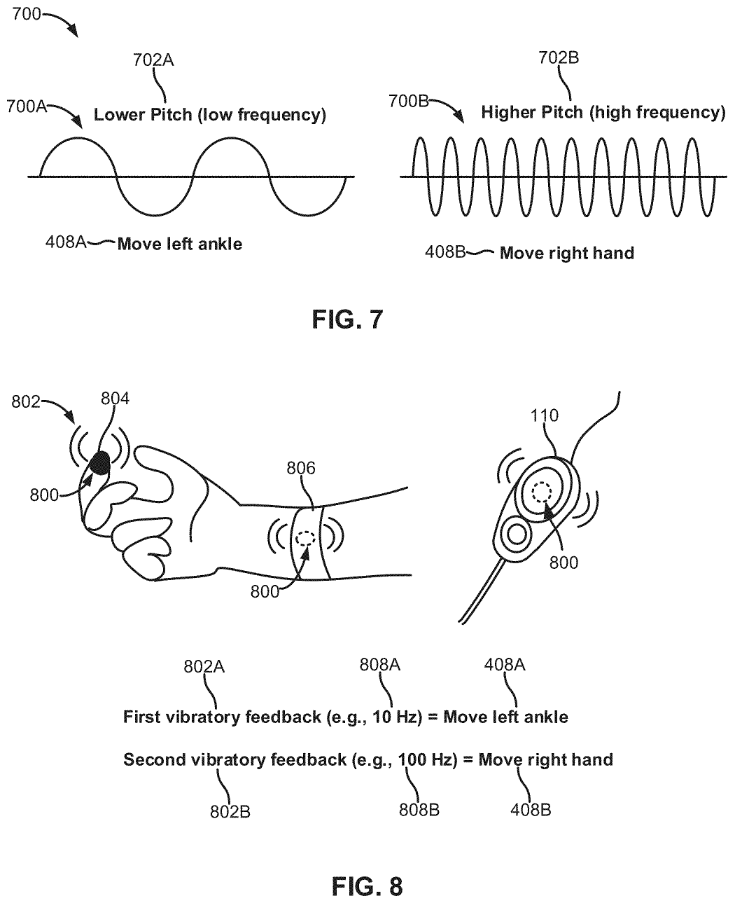

The auditory component 114 can provide neurofeedback to the subject in the form of different sounds 700 (see FIG. 7). In these embodiments, one or more processors of the computing device 106 (see FIG. 1A) can be programmed to execute or carry out instructions stored as part of the neurofeedback module 308 (see FIG. 3) to associate at least a first sound 700A with a first intention 408A of the subject calibrated to certain previously recorded brain activity of the subject and associate a second sound 700B with a second intention 408B of the subject calibrated to certain previously recorded brain activity of the subject.

As shown in FIG. 7, the first sound 700A can be auditorily distinct from the second sound 700B. For example, the first sound 700A can have a first pitch 702A and the second sound 700B can have a second pitch 702B.

The first pitch 702A can be different from the second pitch 702B. For example, the first pitch 702A can be a low-pitched sound (e.g., a frequency of 100 Hz sinusoid) and the second pitch 702B can be a high-pitched sound (e.g., a frequency of 1000 Hz sinusoid). The sounds (e.g., any of the first sound 700A, the second sound 700B, etc.) can be a continuous sound or a sound that maintains a particular pitch (e.g., the first pitch 702A, the second pitch 702B, etc.). In other embodiments contemplated by this disclosure, the pitch of the first sound 700A can be the same as the pitch of the second sound 700B but a volume of the first sound 700A can be different than the volume of the second sound 700B.

In these embodiments, the BCI system 100 can also comprise a user output device communicatively coupled to the computing device 106 and configured to generate a user output to instruct the subject to formulate or carry out an intention 408 (such as moving a left ankle of the subject). The intention 408 can be one of the intentions (e.g., any of the first intention 408A or the second intention 408B) associated with the sounds 700. The subject can then produce brain activity that aligns with or achieves the intention 408 indicated as part of the instruction. This intention 408 can also be referred to as a desired intention of the subject.

In certain embodiments, the user output device can be the auditory component 114 and the user output can be an auditory instruction (e.g., verbal instructions) generated by the auditory component 114 to instruct the subject to formulate or carry out an intention 408. For example, a speaker can play a message to the subject to move the subject's left ankle.

In other embodiments, the user output device can be the display 112 and the user output can be the same text or graphical instructions 412 previously discussed. The text or graphical instructions 412 can be rendered via the display 112.

In response to the instruction (either the auditory instruction or the visual/text instruction), the subject can attempt to produce or activate brain activity to satisfy the instruction. For example, the instruction can be an audio message directed at the subject to move the subject's left ankle. The recording device 101 can then record the current brain activity of the subject and the recorded brain activity can be processed by the pre-processing layer 302 and classified by the classification layer 304 of the decoder module 300.

The classification layer 304 can be trained or calibrated to classify or make predictions concerning the intention 408 of the subject based on previously recorded brain activity. For example, the classification layer 304 can predict the subject's intentions 408 several times per second. The classification layer 304 can be trained using training data collected from the subject.

In some embodiments, the training phase can involve the subject repeatedly initiating, sustaining, and terminating certain thoughts or attempting certain actions while the subject's brain activity is recorded by the recording device 101. For example, one such training session can involve the subject repeatedly resting for 5 seconds followed by attempting to move their left ankle for 5 seconds. The subject's brain activity during this training session can be recorded and the recorded brain activity can be mapped to the subject's intentions 408 to rest and move their left ankle.

The class prediction (i.e., the predicted intention 408 of the subject) can then be fed to the neurofeedback module 308. The neurofeedback module 308 can instruct the auditory component 114 to generate either the first sound 700A or the second sound 700B based on the current brain activity of the subject. For example, the first sound 700A can be a low-pitched sound that is associated with an intention by the subject to move the subject's left ankle and the second sound 700B can be a high-pitched sound that is associated with an intention by the subject to move the subject's right hand. The sounds 700 played by the auditory component 114 can change based on the current brain activity of the subject.

The subject can utilize the auditory feedback to try out different mental activation strategies or focusing techniques to see which works best to achieve a desired sound 700. For example, if the subject was instructed to move their left ankle, the subject can try out different mental activation strategies or focusing techniques until they hear the first sound 700A at the first pitch 702A or to maintain the first sound 700A at the first pitch 702A. In certain embodiments, the subject can begin by hearing a sound 700 with a pitch that is in between the first pitch 702A and the second pitch 702B and their objective can be to try to decrease or increase the pitch of the sound to the first pitch 702A or the second pitch 702B, respectively. In this manner, the auditory feedback generated by the auditory component 114 can provide real-time or near-real-time feedback to the subject concerning how successful they are in self-regulating their brain activity. This, in turn, can provide the subject feedback on how successful they are in controlling the BCI system 100 on command.

In another embodiment, a tactile feedback component 800 can be included as part of the BCI system 100. The tactile feedback component 800 can provide neurofeedback to the subject in the form of tactile feedback 802 (see FIG. 8).

For example, the tactile feedback component 800 can be a wearable component. The tactile feedback component 800 can be communicatively coupled to the computing device 106. As a more specific example, tactile feedback component 800 can be an actuator 804 configured to be worn on a finger of the subject or a wristband or armband actuator 806 configured to be worn on a wrist or arm of the subject. In a further example, the tactile feedback component 800 can be an actuator integrated into the telemetry unit 110 (see FIG. 8).

In these embodiments, one or more processors of the computing device 106 (see FIG. 1A) can be programmed to execute or carry out instructions stored as part of the neurofeedback module 308 (see FIG. 3) to associate at least a first tactile feedback 802A with a first intention 408A of the subject calibrated to certain previously recorded brain activity of the subject and associate a second tactile feedback 802B with a second intention 408B of the subject calibrated to certain previously recorded brain activity of the subject.

As shown in FIG. 8, the first tactile feedback 802A can be different from the second tactile feedback 802B. For example, the first tactile feedback 802A can have a first vibratory frequency 808A and the second tactile feedback 802B can have a second vibratory frequency 808B.

The first vibratory frequency 808A can be different from the second vibratory frequency 808B. For example, the first vibratory frequency 808A can have a low-vibratory frequency (e.g., a frequency of 10 Hz) and the second vibratory frequency 808B can have a higher-vibratory frequency (e.g., a frequency of 100 Hz). The tactile feedback (e.g., any of the first tactile feedback 802A, the second tactile feedback 802B, etc.) can be a continuous vibration or tactile sensation that maintains a particular vibratory frequency (e.g., the first vibratory frequency 808A, the second vibratory frequency 808B, etc.). In other embodiments contemplated by this disclosure, the vibratory frequency of the first tactile feedback 802A can be the same as the vibratory frequency of the second tactile feedback 802B but an intensity or strength of the first tactile feedback 802A can be different than the intensity or strength of the second tactile feedback 802B.

In these embodiments, the BCI system 100 can also comprise a user output device communicatively coupled to the computing device 106 and configured to generate a user output to instruct the subject to formulate or carry out an intention 408 (such as moving a left ankle of the subject). The intention 408 can be one of the intentions 408 (any of the first intention 408A or the second intention 408B) associated with the sounds 700. The subject can then produce brain activity that aligns with or achieves the intention 408 indicated as part of the instruction. This intention 408 can also be referred to as a desired intention of the subject.

In certain embodiments, the user output device can be the auditory component 114 (see, e.g., FIG. 1A) and the user output can be an auditory instruction (e.g., verbal instructions) generated by the auditory component 114 to instruct the subject to formulate or carry out an intention 408. For example, a speaker can play a message to the subject to move the subject's left ankle.

In other embodiments, the user output device can be the display 112 and the user output can be the same text or graphical instructions 412 previously discussed. The text or graphical instructions 412 can be rendered via the display 112.

In response to the instruction (either the auditory instruction or the visual/text instruction), the subject can attempt to produce or activate brain activity to satisfy the instruction. For example, the instruction can be an audio message directed at the subject to move the subject's left ankle. The recording device 101 can then record the current brain activity of the subject and the recorded brain activity can be processed by the pre-processing layer 302 and classified by the classification layer 304 of the decoder module 300.

The classification layer 304 can be trained or calibrated to classify or make predictions concerning the intention 408 of the subject based on previously recorded brain activity. For example, the classification layer 304 can predict the subject's intentions 408 several times per second. The classification layer 304 can be trained using training data collected from the subject.

In some embodiments, the training phase can involve the subject repeatedly initiating, sustaining, and terminating certain thoughts or attempting certain actions while the subject's brain activity is recorded by the recording device 101. For example, one such training session can involve the subject repeatedly resting for 5 seconds followed by attempting to move their left ankle for 5 seconds. The subject's brain activity during this training session can be recorded and the recorded brain activity can be mapped to the subject's intentions 408 to rest and move their left ankle.

The class prediction (i.e., the predicted intention 408 of the subject) can then be fed to the neurofeedback module 308. The neurofeedback module 30 can instruct the tactile feedback component 800 to generate either the first tactile feedback 802A or the second tactile feedback 802B based on the current brain activity of the subject. For example, the first tactile feedback 802A can be tactile feedback at a low vibratory frequency that is associated with an intention by the subject to move the subject's left ankle and the second tactile feedback 802B can be tactile feedback at a higher vibratory frequency that is associated with an intention by the subject to move the subject's right hand. The tactile feedback generated by the tactile feedback component 800 can change based on the current brain activity of the subject.

The subject can utilize the tactile feedback to try out different mental activation strategies or focusing techniques to see which works best to achieve a desired tactile feedback 802. For example, if the subject was instructed to move their left ankle, the subject can try out different mental activation strategies or focusing techniques until they feel the first tactile feedback 802A at the first vibratory frequency 808A or to maintain the first tactile feedback 802A at the first vibratory frequency 808A. In certain embodiments, the subject can begin by feeling tactile feedback with a vibratory frequency that is in between the first vibratory frequency 808A and the second vibratory frequency 808B and their objective can be to try to decrease or increase the vibratory frequency to the first vibratory frequency 808A or the second vibratory frequency 808B, respectively. In this manner, the tactile feedback generated by the tactile feedback component 800 can provide real-time or near-real-time feedback to the subject concerning how successful they are in self-regulating their brain activity. This, in turn, can provide the subject feedback on how successful they are in controlling the BCI system 100 on command.

Although FIGS. 7 and 8 discuss auditory feedback at two different sound frequencies and tactile feedback at two different vibratory frequencies, it is contemplated by this disclosure that the BCI system 100 can generate any number of sounds of different sound frequencies and any number of tactile feedback at different vibratory frequencies. The number or types of sounds/sound frequencies or the number or types of tactile feedback/vibratory frequencies can match the number of possible class predictions or possible intentions 408.

A number of embodiments have been described. Nevertheless, it will be understood by one of ordinary skill in the art that various changes and modifications can be made to this disclosure without departing from the spirit and scope of the embodiments. Elements of systems, devices, apparatus, and methods shown with any embodiment are exemplary for the specific embodiment and can be used in combination or otherwise on other embodiments within this disclosure. For example, the steps of any methods depicted in the figures or described in this disclosure do not require the particular order or sequential order shown or described to achieve the desired results. In addition, other steps operations may be provided, or steps or operations may be eliminated or omitted from the described methods or processes to achieve the desired results. Moreover, any components or parts of any apparatus or systems described in this disclosure or depicted in the figures may be removed, eliminated, or omitted to achieve the desired results. In addition, certain components or parts of the systems, devices, or apparatus shown or described herein have been omitted for the sake of succinctness and clarity.

Accordingly, other embodiments are within the scope of the following claims and the specification and/or drawings may be regarded in an illustrative rather than a restrictive sense.

Each of the individual variations or embodiments described and illustrated herein has discrete components and features which may be readily separated from or combined with the features of any of the other variations or embodiments. Modifications may be made to adapt a particular situation, material, composition of matter, process, process act(s) or step(s) to the objective(s), spirit or scope of the present invention.

Methods recited herein may be carried out in any order of the recited events that is logically possible, as well as the recited order of events. Moreover, additional steps or operations may be provided or steps or operations may be eliminated to achieve the desired result.

Furthermore, where a range of values is provided, every intervening value between the upper and lower limit of that range and any other stated or intervening value in that stated range is encompassed within the invention. Also, any optional feature of the inventive variations described may be set forth and claimed independently, or in combination with any one or more of the features described herein. For example, a description of a range from 1 to 5 should be considered to have disclosed subranges such as from 1 to 3, from 1 to 4, from 2 to 4, from 2 to 5, from 3 to 5, etc. as well as individual numbers within that range, for example 1.5, 2.5, etc. and any whole or partial increments therebetween.

All existing subject matter mentioned herein (e.g., publications, patents, patent applications) is incorporated by reference herein in its entirety except insofar as the subject matter may conflict with that of the present invention (in which case what is present herein shall prevail). The referenced items are provided solely for their disclosure prior to the filing date of the present application. Nothing herein is to be construed as an admission that the present invention is not entitled to antedate such material by virtue of prior invention.

Reference to a singular item, includes the possibility that there are plural of the same items present. More specifically, as used herein and in the appended claims, the singular forms “a,” “an,” “said” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. Unless defined otherwise, all technical and scientific terms used herein have the same meaning as commonly understood by one of ordinary skill in the art to which this invention belongs.

Reference to the phrase “at least one of”, when such phrase modifies a plurality of items or components (or an enumerated list of items or components) means any combination of one or more of those items or components. For example, the phrase “at least one of A, B, and C” means: (i) A; (ii) B; (iii) C; (iv) A, B, and C; (v) A and B; (vi) B and C; or (vii) A and C.

In understanding the scope of the present disclosure, the term “comprising” and its derivatives, as used herein, are intended to be open-ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives. Also, the terms “part,” “section,” “portion,” “member” “element,” or “component” when used in the singular can have the dual meaning of a single part or a plurality of parts. As used herein, the following directional terms “forward, rearward, above, downward, vertical, horizontal, below, transverse, laterally, and vertically” as well as any other similar directional terms refer to those positions of a device or piece of equipment or those directions of the device or piece of equipment being translated or moved.

Finally, terms of degree such as “substantially”, “about” and “approximately” as used herein mean the specified value or the specified value and a reasonable amount of deviation from the specified value (e.g., a deviation of up to ±0.1%, ±1%, ±5%, or ±10%, as such variations are appropriate) such that the end result is not significantly or materially changed. For example, “about 1.0 cm” can be interpreted to mean “1.0 cm” or between “0.9 cm and 1.1 cm.” When terms of degree such as “about” or “approximately” are used to refer to numbers or values that are part of a range, the term can be used to modify both the minimum and maximum numbers or values.

This disclosure is not intended to be limited to the scope of the particular forms set forth, but is intended to cover alternatives, modifications, and equivalents of the variations or embodiments described herein. Further, the scope of the disclosure fully encompasses other variations or embodiments that may become obvious to those skilled in the art in view of this disclosure.

1. A brain-computer interface system, comprising:
a recording device configured to record a brain activity of a subject;
a computing device communicatively coupled to the recording device, wherein the computing device comprises one or more processors programmed to:
construct a neurofeedback graphical user interface (GUI) comprising a plurality of graphic portions including at least a first graphic portion representing a first intention of the subject calibrated to certain previously recorded brain activity of the subject and a second graphic portion representing a second intention of the subject calibrated to certain other previously recorded brain activity of the subject, and
construct a graphic element appearing in at least one of the first graphic portion and the second graphic portion, wherein the graphic element represents a current brain activity of the subject recorded by the recording device, and wherein the graphic element is moveable between the first graphic portion and the second graphic portion; and
a display, communicatively coupled to the computing device, configured to display the neurofeedback GUI and the graphic element to the subject to aid the subject in producing brain activity that aligns with a desired intention of the subject.

2. The system of claim 1, wherein the desired intention is one of the first intention or the second intention.

3. The system of claim 1, wherein the brain activity of the subject recorded by the recording device are reduced to univariate data, wherein the univariate data is presented in a one-dimensional space through the neurofeedback GUI, wherein the first graphic portion comprises a first segment of the one-dimensional space, and wherein the second graphic portion comprises a second segment of the one-dimensional space.

4. The system of claim 3, wherein the one-dimensional space is a number line and the graphic element is a dot moveable along the number line.

5. The system of claim 1, wherein the brain activity of the subject recorded by the recording device is collected as multivariate data, wherein the multivariate data is presented in a two-dimensional space through the neurofeedback GUI, wherein the first graphic portion comprises a first area of the two-dimensional space, and wherein the second graphic portion comprises a second area of the two-dimensional space.

6. The system of claim 5, wherein the two-dimensional space is a graph having two axes.

7. The system of claim 6, wherein the multivariate data is reduced into the two-dimensional space using a dimensionality reduction function.

8. The system of claim 6, wherein the multivariate data is reduced into the two-dimensional space using principal component analysis (PCA).

9. The system of claim 8, wherein the first area and the second area of the two-dimensional space are positioned on the graph by projecting coordinates within the two-dimensional space back to a multi-dimensional space corresponding to the multivariate data.

10. The system of claim 6, wherein the multivariate data is reduced into the two-dimensional space using a hyperplane of a linear classifier.

11. The system of claim 6, wherein the multivariate data is reduced into the two-dimensional space using a non-linear dimensionality reduction function.

12. The system of claim 6, wherein the graphic element is a dot moveable within the graph.

13. The system of claim 1, wherein the first graphic portion is presented in a first color and the second graphic portion is presented in a second color.

14. The system of claim 1, wherein the brain activity recorded is a power of a neural oscillation or brainwave of the subject or a change in blood flow within the brain of the subject.

15. The system of claim 1, wherein the recording device is at least one of an implantable recording device, an electroencephalography (EEG) device, an electrocorticography (ECoG) device, a functional magnetic resonance imaging (fMRI) machine, and a functional near infrared spectroscopy (fNIRS) device.

16. The system of claim 1, wherein either the first intention or the second intention is an intention of the subject to move a body part of the subject.

17. The system of claim 1, wherein either the first intention or the second intention is achieving or maintaining a neural rest state.

18. The system of claim 1, wherein a size of the graphic element displayed is configured to change in accordance with a certainty value associated with a positioning of the graphic element within at least one of the first graphic portion and the second graphic portion based on the current brain activity of the subject recorded by the recording device.

19. The system of claim 1, wherein a color intensity of the graphic element displayed is configured to change in accordance with a certainty value associated with a positioning of the graphic element within at least one of the first graphic portion and the second graphic portion based on the current brain activity of the subject recorded by the recording device.

20. The system of claim 1, wherein the graphic element comprises a plurality of smaller graphic icons and wherein a density or shape of the smaller graphic icons are configured to change in accordance with a certainty value associated with a positioning of the graphic element within at least one of the first graphic portion and the second graphic portion based on the current brain activity of the subject recorded by the recording device.

21. A brain-computer interface system, comprising:
a recording device configured to record a brain activity of a subject;
a computing device communicatively coupled to the recording device, wherein the computing device comprises one or more processors programmed to:
associate a first graphic element with a first intention of the subject calibrated to certain previously recorded brain activity of the subject and associate a second graphic element with a second intention of the subject calibrated to certain previously recorded brain activity of the subject, wherein the first graphic element is visually distinct from the second graphic element;
a display, communicatively coupled to the computing device, configured to display instances of either the first graphic element or the second graphic element in temporal succession based on a current brain activity of the subject recorded by the recording device in order to aid the subject in producing brain activity that align with a desired intention of the subject, wherein the desired intention is either the first intention or the second intention.

22.-24. (canceled)

25. A brain-computer interface system, comprising:
a recording device configured to record a brain activity of a subject;
a computing device communicatively coupled to the recording device, wherein the computing device comprises one or more processors programmed to:
associate a first sound with a first intention of the subject calibrated to certain previously recorded brain activity of the subject and associate a second sound with a second intention of the subject calibrated to certain previously recorded brain activity of the subject, wherein the first sound is auditorily distinct from the second sound;
a user output device, communicatively coupled to the computing device, configured to generate a user output to instruct the subject to formulate or carry out a desired intention of the subject, wherein the desired intention is either the first intention or the second intention;
an auditory component, communicatively coupled to the computing device, configured to generate either the first sound or the second sound based on a current brain activity of the subject recorded by the recording device in order to aid the subject in producing brain activity that align with the desired intention of the subject.

26. (canceled)

27. (canceled)

28. A brain-computer interface system, comprising:
a recording device configured to record a brain activity of a subject;
a computing device communicatively coupled to the recording device, wherein the computing device comprises one or more processors programmed to:
associate a first tactile feedback with a first intention of the subject calibrated to certain previously recorded brain activity of the subject and associate a second tactile feedback with a second intention of the subject calibrated to certain previously recorded brain activity of the subject, wherein the first tactile feedback is sensorially distinct from the second tactile feedback;
a user output device, communicatively coupled to the computing device, configured to generate a user output to instruct the subject to formulate or carry out a desired intention of the subject, wherein the desired intention is either the first intention or the second intention;
a tactile feedback component, communicatively coupled to the computing device, configured to generate either the first tactile feedback or the second tactile feedback based on a current brain activity of the subject recorded by the recording device in order to aid the subject in producing brain activity that align with the desired intention of the subject.

29. (canceled)

30. (canceled)

31. A method of conducting neurofeedback training, comprising:
recording a brain activity of a subject using a recording device;
constructing a neurofeedback graphical user interface (GUI) using one or more processors of a computing device communicatively coupled to the recording device, wherein the neurofeedback GUI comprises a plurality of graphic portions including at least a first graphic portion representing a first intention of the subject calibrated to certain previously recorded brain activity of the subject and a second graphic portion representing a second intention of the subject calibrated to certain other previously recorded brain activity of the subject;
constructing a graphic element appearing in at least one of the first graphic portion and the second graphic portion, wherein the graphic element represents a current brain activity of the subject recorded by the recording device, and wherein the graphic element is moveable between the first graphic portion and the second graphic portion; and
displaying the neurofeedback GUI and the graphic element to the subject via a display communicatively coupled to the computing device to aid the subject in producing brain activity that aligns with a desired intention of the subject.

32.-50. (canceled)

51. A method of conducting neurofeedback training, comprising:
recording a brain activity of a subject using a recording device;
associating, using one or more processors of a computing device communicatively coupled to the recording device, a first graphic element with a first intention of the subject calibrated to certain previously recorded brain activity of the subject and associate a second graphic element with a second intention of the subject calibrated to certain previously recorded brain activity of the subject, wherein the first graphic element is visually distinct from the second graphic element;
constructing a neurofeedback graphical user interface (GUI) using the one or more processors of the computing device;
displaying, via a display communicatively coupled to the computing device, the neurofeedback GUI, wherein instances of either the first graphic element or the second graphic element are rendered in temporal succession as part of the neurofeedback GUI based on a current brain activity of the subject recorded by the recording device in order to aid the subject in producing brain activity that align with a desired intention of the subject, wherein the desired intention is either the first intention or the second intention.

52.-54. (canceled)

55. A method of conducting neurofeedback training, comprising:
recording a brain activity of a subject using a recording device;
associating, using one or more processors of a computing device communicatively coupled to the recording device, a first sound with a first intention of the subject calibrated to certain previously recorded brain activity of the subject and associate a second sound with a second intention of the subject calibrated to certain previously recorded brain activity of the subject, wherein the first sound is auditorily distinct from the second sound;
generating, using a user output device communicatively coupled to the computing device, a user output to instruct the subject to formulate or carry out a desired intention of the subject, wherein the desired intention is either the first intention or the second intention;
generating, using an auditory component communicatively coupled to the computing device, either the first sound or the second sound based on a current brain activity of the subject recorded by the recording device in order to aid the subject in producing brain activity that align with the desired intention of the subject.

56. (canceled)

57. (canceled)

58. A method of conducting neurofeedback training, comprising:
recording a brain activity of a subject using a recording device;
associating, using one or more processors of a computing device communicatively coupled to the recording device, a first tactile feedback with a first intention of the subject calibrated to certain previously recorded brain activity of the subject and associate a second tactile feedback with a second intention of the subject calibrated to certain previously recorded brain activity of the subject, wherein the first tactile feedback is sensorially distinct from the second tactile feedback;
generating, using a user output device communicatively coupled to the computing device, a user output to instruct the subject to formulate or carry out a desired intention of the subject, wherein the desired intention is either the first intention or the second intention;
generating, using a tactile feedback component communicatively coupled to the computing device, either the first tactile feedback or the second tactile feedback based on a current brain activity of the subject recorded by the recording device in order to aid the subject in producing brain activity that align with the desired intention of the subject.

59. (canceled)

60. (canceled)
